(19) United States
(12) Patent Application Publication
(10) Pub. No.: US 2010/0297651 A1
(43) Pub. Date: Nov. 25, 2010

(54) Methods of Modulating Vesicular Trafficking

(75) Inventors: Vladimir Marshansky, Sharon, MA (US); Maria Merkulova, Arlington, MA (US)

Correspondence Address: FISH & RICHARDSON PC, P.O. BOX 1022, MINNEAPOLIS, MN, 55440-1022, US

(73) Assignee: THE GENERAL HOSPITAL CORPORATION, Boston, MA (US)

(21) Appl. No.: 12/785,139

(22) Filed: May 21, 2010

Related U.S. Application Data
(60) Provisional application No. 61/180,277, filed on May 21, 2009.
(60) Provisional application No. 61/301,873, filed on Feb. 5, 2010.

(51) Int. Cl. C12Q 1/68; C07K 7/08

(52) U.S. Cl. 435/6; 530/326

(57) ABSTRACT

Described are methods of identifying compounds useful for modulating vesicular trafficking, particularly those that disrupt the interactions between ARNO and the V-ATPase a2-subunit. Also described are peptides that modulate vesicular trafficking.

### CLAIM OF PRIORITY

This application claims the benefit of U.S. Provisional Patent Application Ser. Nos. 61/180,277, filed on May 21, 2009, and 61/301,873, filed on Feb. 5, 2010, the entire contents of which are hereby incorporated by reference.

### FEDERALLY SPONSORED RESEARCH OR DEVELOPMENT

This invention was made with Government support under Grant No. DK038452 awarded by the National Institutes of Health. The Government has certain rights in the invention.

### TECHNICAL FIELD

This invention relates to methods of identifying compounds useful for modulating vesicular trafficking, particularly those that disrupt the interactions between ARNO and the V-ATPase a2-subunit. Also described are peptides that modulate vesicular trafficking.

### BACKGROUND

Vesicular trafficking is an essential cellular process in eukaryotic cells to deliver either membrane proteins or soluble cargos from one compartment to another (see, e.g., Marshansky and Futai, Curr. Op. Cell Biol. 20:415-426 (2008)). Defects of the trafficking within exocytotic and endocytotic pathways, including defective V-ATPase function, have been recently recognized as an important cell biological mechanism in various human diseases including cancer, neurological disorders, and autoimmune and metabolic diseases including diabetes among others (Aridor and Hannan Traffic 2000, 1:836-851; Aridor and Hannan Traffic 2002, 3:781-790).

### SUMMARY

The present invention is based, at least in part, on the discovery of the regions of a2-subunit V-ATPase and ARNO that interact, methods of screening using isolated interacting domains, and an a2-subunit derived peptide that is capable of inhibiting the function of the enzymatic GEF-activity of ARNO within V-ATPase/small GTPase complex. This peptide can be used as a therapeutic agent in the treatment of a number of conditions, including viral infection and cancer metastasis.

Thus, in one aspect the invention provides isolated peptides of SEQ ID NO:27, e.g., peptides consisting essentially of amino acids 1-17 of a2-subunit V-ATPase; in some embodiments, up to an additional about 402 amino acids of a2-subunit V-ATPase can be added, e.g., up to 100, up to 200, up to 300, up to 385, so long as the peptides with the additional amino acids retain one, two, or all of the following activities: i) high affinity interaction with Sec7 domain of ARNO; ii) modulating enzymatic ARNO's GEF-activity in GTP/GDP-exchange assay; iii) targeting to the endosomal/lysosomal pathway, and iv) modulating function of this pathway in living cells.

Thus in some embodiments the peptides are isolated a2N-derived peptides including: i) a2N-03 (or a2N₃₅₋₄₉); ii) a2N-11 (or a2N₁₉₈₋₂₁₄); iii) a2N-12 (or a2N₂₁₅₋₂₃₀); iv) a2N-18 (or a2N₃₁₃₋₃₃₁) and v) a2N-22 (or a2N₃₈₆₋₄₀₂). Each of these peptides is capable of interaction with ARNO in pull-down experiments. In some embodiments, the peptide is a recombinant a2N (or a2N₁₋₄₀₂), a2N-N (or a2N₁₋₁₃₃) and a2N-C (or a2N₁₃₄₋₃₉₃) polypeptide, each of which is capable of high-affinity interaction with full-length ARNO in BIAcore and pull-down assays and modulating enzymatic ARNO's GEF-activity in GTP/GDP-exchange assay. These numbers are relative to SEQ ID NOs. 28 or 30.

In another aspect, the invention provides fusion peptides comprising (i) a vacuolar-type H+-ATPase (V-ATPase)-derived sequence consisting essentially of SEQ ID NO:27 and (ii) a non-V-ATPase sequence, e.g., a detectable label or a cell-penetrating peptide.

In yet a further aspect, the invention features methods for identifying candidate compounds that inhibit binding of a2-subunit V-ATPase to ARF nucleotide-binding site opener (ARNO) (also known as cytohesin-2). The methods include providing a test sample, e.g., a cell-free test sample, including (i) an ARNO interacting domain comprising Sec7 region of ARNO, e.g., full-length ARNO or an isolated ARNO Sec7 domain; and (ii) an a2-subunit V-ATPase interacting domain comprising an isolated a2N peptide or ARNO-binding fragment thereof (e.g., a2N (or a2N₁₋₄₀₂), a2N-N (or a2N₁₋₁₃₃), a2N-C (or a2N₁₃₄₋₃₉₃) or a2N-01 (or a2N₁₋₁₇) domains);

contacting the test sample with a test compound;

determining a level of binding between the interacting domains in the presence of the test compound; and

comparing the level of binding between the interacting domains to a level of binding in the absence of the test compound, e.g., in the same sample or in a control sample, or a reference level determined previously, wherein a decrease in binding in the presence of the test compound means that the test compound is a candidate compound that inhibits binding of a2-subunit V-ATPase to ARNO.

In some embodiments, the a2-subunit V ATPase interacting domain comprises a2N-01 (or a2N₁₋₁₇).

The level of binding can be determined using methods known in the art, e.g., plasmon resonance binding. In some embodiments, e.g., wherein the interacting domains each further comprise a fluorescent moiety, the level of binding is determined using fluorescence polarization.

In some embodiments, the methods further include selecting a candidate compound that inhibits binding of a2-subunit V-ATPase to ARNO; providing a cell that expresses a functional a2-subunit V-ATPase/ARNO complex e.g., endogenous or exogenous complex, e.g., MTC, HeLa and HEK kidney cells as well as in MCF7 and MDA-MB231 breast cancer cells; contacting the cell with the selected candidate compound; evaluating activity of one or both of the a2-subunit V-ATPase and ARNO in the cell, and selecting as a candidate therapeutic compound a candidate compound that inhibits activity of one or both of the ARNO and a2-subunit V-ATPase in the cell.

In some embodiments, ARNO's GEF activity with Arf6 is assayed using a radiolabel-based GDP/GTP-exchange assay. In some embodiments, vesicular trafficking and function of endosomal/lysosomal protein degradation pathway is modified by a2-subunit V-ATPase derived peptide and inhibition of ARNO's enzymatic GDP/GTP-exchange activity using megalin/cubilin-mediated uptake of labeled albumin, e.g., albumin-Alexa555, in a fluorimetric cell-population assay.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic illustration of the design of a2N-derived peptides according to their secondary structure predicted by PSIPRED and COILS software. The α-helices are shown as grey cylinders, β-strands are shown as lighter grey arrows.

FIG. 2A is an image of a Western blot showing the results of an a2N-derived peptide pull-down assay. The indicated a2N-derived peptides were immobilized on streptavidin-beads and incubated with purified recombinant GST-ARNO(wt). Interacting complexes were eluted and analyzed by Western blotting using monoclonal anti-GST antibodies.

FIG. 2B is an image of a Western blot showing the results of a pull-down assay with selected a2N-derived interacting peptides incubated with either GST (negative control) or GST-ARNO(wt). The first two lanes show the patterns obtained when either GST-only (lane 1) or GST-ARNO(wt) (lane 2) were loaded onto the gel. The a2N-derived peptides were used in a pull-down assay as described above and show specific interaction with GST-ARNO(wt) but not with GST-only.

FIG. 2C is an image of a Western blot showing that both the amino terminus (a2N-N) and carboxyl terminus (a2N-C) of the cytosolic tail of the a2-subunit interact with ARNO(wt). Left panel, a pull-down (PD) of in vitro translated metabolically labeled a2N-N (a2N₁₋₁₃₃) with recombinant GST-only and GST-ARNO(wt) used as a baits. Autoradiography (AR) showing the direct interaction of a2N-N-[³⁵S] with GST-ARNO(wt). Right panel, a pull-down (PD) of in vitro translated metabolically labeled a2N-C (a2N₁₃₄₋₃₉₃) with recombinant GST-only and GST-ARNO(wt) used as a baits. Autoradiography (AR) showing the direct interaction of a2N-C-[³⁵S] with GST-ARNO(wt).

FIG. 3 is an image of a gel showing the purity of GST and 6His double-tagged ARNO(wt) and its domains used in GST pull-down experiments. Proteins were resolved by NuPAGE and stained by Ponceau-S. Molecular weight markers are shown on the right.

FIG. 4A is a table showing names, position, length and sequence of synthetic ARNO-derived peptides, as follows: i) Sec7-PH-linker; ii) non-phosphorylated PB-domain (ARNO₃₇₅₋₄₀₀) and iii) phosphorylated PB-domain domain (ARNO₃₇₅₋₄₀₀^{P}). PKC-dependent phosphorylation of serine-392 (Ser₃₉₂) of ARNO is indicated.

FIGS. 4B-C are images of Western blots showing (4B) the results of peptide pull-down assays for interaction of a2N with Sec7-PH-linker of ARNO and (4C) phosphorylation-dependent interaction of a2N with ARNO-derived peptide corresponding to the PB-domain of ARNO. Corresponding ARNO-derived peptides were immobilized on streptavidin-beads and incubated with in vitro translated recombinant a2N. Interacting complexes were eluted and analyzed by Western blotting using anti-a2N antibodies.

FIGS. 5A-C are a set of three images of Western blots showing the identification of interaction motif of a2N that is involved in specific binding with the Sec7 domain of ARNO. 5A, The catalytic Sec7 domain of ARNO interacts with two a2N-derived peptides, a2N-01 and a2N-03, in pull-down experiments. The peptide pull-down assay was performed with six a2N-derived interaction peptides immobilized on streptavidin-beads and incubated with purified recombinant GST-only or GST-Sec7 domain. Interacting complexes were eluted and analyzed by Western blotting using monoclonal anti-GST antibodies as described below. The loading controls for GST-only (lane 1) or GST-Sec7 (lane 2) are shown. 5B, 5C: Peptide a2N-01 specifically interacts with Sec7 but not PH-domain (5B) or PB-domain (5C) of ARNO. The peptide pull-down assay was performed with peptides a2N-01 and a2N-03 immobilized on streptavidin-beads and incubated with purified recombinant GST-PH domain (5B) or GST-PB domain (5C). Interacting complexes were eluted and analyzed by Western blotting using monoclonal anti-GST antibodies. The loading control of GST-PH-domain (5B) or GST-PB-domain (5C) is shown on the left.

FIGS. 6A-6B are a pair of line graphs showing estimation of the binding affinities for the specific interaction domains between V-ATPase a2-subunit and ARNO. 6A, Kinetic analysis and estimation of a dissociation constant (K_{D}) for interaction between full-length a2N(wt) and ARNO(wt) proteins. Sensorgrams for the binding of recombinant a2N-6His immobilized as ligand on the sensor chip, with various concentrations of 6His-ARNO as analyte are shown as follows. The responses on the sensorgram are designated in resonance units (RU). The best fitted curves with bivalent analyte interaction isotherm were obtained and the K_{D}=3.13×10⁻⁷M value was determined using BIAcore T100″ evaluation software. 6B, Kinetic analysis and estimation of K_{D} for interaction between a2N-01 peptide and Sec7-domain of ARNO. Sensorgrams for the binding of a2N-01 peptide immobilized as ligand on the sensor chip, with various concentrations of Sec7-domain as analyte are shown as follows. The responses on the sensorgram are designated in resonance units (RU). The best fitted curves with single 1:1 interaction isotherm were obtained and the K_{D}=3.44×10⁻⁷M value was determined using BIAcore™ T100 evaluation software.

FIGS. 7A-B are a pair of fluorescence photomicrographs showing differential delivery of cell-penetrating peptides FITC-TAT (7A) and FITC-a2N-01-TAT in mouse tubule cells after 10 minutes of incubation.

FIG. 8 is a line graph showing the time-course of GEF activity of ARNO with Arf6 in presence of GTPγS and phospholipid vesicles containing various amounts of PIP2.

FIG. 9 is a line graph showing specific inhibition of ARNO's GEF activity with Arf6 by synthetic peptide FITC-a2N-01-TAT in presence of 0.5% PIP2 containing phospholipid vesicles.

FIG. 10 is a line graph showing potent and specific inhibition of ARNO's GEF activity with Arf6 by synthetic peptide FITC-a2N-01-TAT in presence of 5% PIP2 containing phospholipid vesicles.

FIG. 11 is a line graph showing that vesicular trafficking within the endosomal/lysosomal protein degradation pathway in mouse proximal tubules cells in vivo is greatly modified by FITC-a2N-01-TAT but not FITC-TAT peptide.

FIG. 12 is a sequence alignment of the human (SEQ ID NO:28) and mouse (SEQ ID NO:30) a2-subunit V-ATPase, with a consensus sequence (SEQ ID NO:29) in between. +, conservative substitution.

### DETAILED DESCRIPTION

The present invention is based, at least in part, on the discovery of the regions of the a2-subunit V-ATPase and ARNO that interact, methods of screening using isolated interacting domains, and a peptide that is capable of inhibiting of the enzymatic GEF-activity of ARNO and modulating V-ATPase function within the V-ATPase/small GTPase complex. This peptide can be used as a therapeutic agent in the treatment of a number of conditions as described herein.

Vacuolar-Type H⁺-ATPase (V-ATPase)

The Vacuolar-type H⁺-ATPase (V-ATPase) is a proton pumping nano-motor involved in acidification of variety intracellular organelles and extracellular compartments. It is absolutely essential for vesicular trafficking along both exocytotic and endocytotic pathways of eukaryotic cells. Deficient function of V-ATPase and defects of vesicular acidification have been also recently recognized as important mechanisms of variety of human diseases and discussed below. Consistent with the presence of V-ATPases in diverse compartments a large spectrum of subunit isoforms were found (Aridor and Hannan Traffic 2000, 1:836-851; Aridor and Hannan Traffic 2002, 3:781-790. Marshansky and Futai, Curr Opin Cell Biol 2008, 20:415-426; Grüeber and Marshansky, BioEssays 2008, 30: 1096-1099; Hurtado-Lorenzo et al., Nature Cell Biol 2006, 6:124-136; Forgac, Nat. Rev. Mol. Cell. Biol. 2007, 11: 917-29; Hinton et al., Pflugers Arch. 2009, 457: 589-598). The expression of these isoforms is tissue and cell specific. Recent experiments demonstrated that targeting of V-ATPases with unique combinations of subunit isoforms are localized in specific cellular membranes which could be dictated by their functions (Wagner et al., Physiol. Rev. 2004, 84:1263-1314; Marshansky and Futai, Curr Opin Cell Biol 2008, 20:415-426; Grüeber and Marshansky, BioEssays 2008, 30: 1096-1099). Intracellular targeting of V-ATPase is regulated by a-subunit isoforms. Four a-subunit isoforms (a1, a2, a3 and a4) were found in mice and humans (Toyomura et al., J Biol Chem 2000, 275: 8760-8765; Oka et al., J Biol Chem 2001, 276: 40050-40054; Smith et al., J Biol Chem 2001, 276: 42382-42388; Nishi and Forgac, J Biol Chem 2000, 275: 6824-6830). They are localized in different endomembrane organelles and plasma membrane of specialized cells (Wagner et al., Physiol. Rev. 2004, 84:1263-1314; Marshansky and Futai, Curr Opin Cell Biol 2008, 20:415-426; Grüeber and Marshansky, BioEssays 2008, 30: 1096-1099).

The sequence of human a2-subunit V-ATPase is available in the GenBank database under Accession No. NM_{—}012463.3 (nucleic acid) and NP_{—}036595.2 (amino acid).

a2N-01 Peptides

Described herein are a2N-01 peptides having the sequence MGSLFRSE[T/S]MCLAQLFL (SEQ ID NO:27). These peptides are useful, e.g., as therapeutic agents and as a binding domain for use in the screening methods described herein. The peptides can be synthesized using peptide synthesis methods known in the art, or can be recombinant (e.g., expressed in a cell or animal and isolated and/or purified therefrom using methods known in the art). In some embodiments, the peptide has an amino acid sequence that at least 80%, 85%, 90%, 95%, 98% or more identical to SEQ ID NO:27. In some embodiments, the peptide has the sequence MGSLFRSESMCLAQLFL (SEQ ID NO:1, from mouse a2-subunit V-ATPase, GenBank Acc Nos. BAA93007.1). In some embodiments, the peptide has the sequence MGSLFRSETMCLAQLFL (SEQ ID NO:26, from human a2-subunit V-ATPase, GenBank Acc. Nos. NP 036595.2). In some embodiments, the peptide has an amino acid sequence with a substitution other than T or S at amino acid 9 of SEQ ID NO:27.

The a2N-01 peptides can be part of a fusion protein, i.e., can be linked to a non-V-ATPase sequence. The non-V-ATPase sequence can be, e.g., a cell penetrating peptide or a label, e.g., a fluorescent protein such as GFP or a variant thereof. The non-V-ATPase polypeptide can be fused to the N-terminus or C-terminus of the a2N-01 polypeptide.

The fusion protein can include a moiety which has a high affinity for a ligand. For example, the fusion protein can be a GST-a2N-01 fusion protein in which the a2N-01 sequences are fused to the C-terminus of the GST sequences. Such fusion proteins can facilitate the purification of recombinant a2N-01. Alternatively, the fusion protein can be a a2N-01 protein containing a heterologous signal sequence at its N-terminus. In certain host cells (e.g., mammalian host cells), expression and/or secretion of a2N-01 can be increased through use of a heterologous signal sequence.

In some embodiments, the fusion protein includes a cell-penetrating peptide (CPP) sequence that facilitates delivery of the a2N-01 peptide to the intracellular space, e.g., HIV-derived TAT peptide, penetratins, transportans, or hCT derived cell-penetrating peptides, see, e.g., Caron et al., (2001) Mol. Ther. 3(3):310-8; Langel, Cell-Penetrating Peptides: Processes and Applications (CRC Press, Boca Raton Fla. 2002); El-Andaloussi et al., (2005) Curr Pharm Des. 11(28):3597-611; and Deshayes et al., (2005) Cell Mol Life Sci. 62(16):1839-49.

Fusion proteins can include all or a part of a serum protein, e.g., an IgG constant region, or human serum albumin.

Expression vectors are commercially available that already encode a fusion moiety (e.g., a GST polypeptide). An a2N-01 peptide-encoding nucleic acid can be cloned into such an expression vector such that the fusion moiety is linked in-frame to the a2N-01 peptide.

In some embodiments, the peptide is coupled (i.e., physically linked) to a detectable substance (i.e., labelled). Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

The a2N-01 peptides and fusion proteins can be incorporated into pharmaceutical compositions and administered to a subject in vivo. a2N-01 peptides and fusion proteins can be useful therapeutically for the treatment of the diseases described herein, e.g., i) cancer treatment and inhibition of metastatic invasion, ii) control of viral and bacterial infection, iii) bones degenerative diseases; iv) lysosomal-storage diseases; v) neurodegenerative diseases associated with abnormal autophagy (including Alzheimer's, Parkinson's and Huntington's); vi) metabolic diseases such as diabetes and obesity with their organ specific complications (including nephropathy and cardiopathy); vii) kidney specific diseases (including Fanconi syndrome and Dent's disease); and viii) prevention and control of accumulation of toxic substances and/or drugs in human tissues including kidney among other disorders that would benefit from reduced ARNO and V-ATPase activity

Moreover, the a2N-01 peptides and fusion proteins can be used as immunogens to produce anti-V-ATPase antibodies in a subject, and in screening assays to identify molecules which inhibit the interaction of V-ATPase and ARNO, as described herein.

Role of V-ATPase/ARNO/Arf6 Complex in Trafficking of β1-Integrin and Modulation of Cell Adhesion, Motility and Invasiveness.

Cell adhesion and motility are crucial for cell functions including tissue formation, maintenance and repair. Migration of the cells involves the variety of cell biological processes including: i) cytoskeleton rearrangement; ii) endocytosis and recycling of signaling molecules (such as growth receptors) as well as adhesion molecules (such as integrins) among others. Targeting of the pathways which are controlling cell migration are of particular importance in basic and clinical studies aiming to prevent tumor metastasis and promote cancer therapy. Recently, major advances have been made to uncover fundamental cell biological mechanisms involved in regulation of cell adhesion and motility. The interplay between Arf-family small GTPases, their regulatory GEF-proteins, V-ATPase and integrins in epithelial cell adhesion, migration and in cancer cell invasion is emerging and discussed below.

ARNO/Arf6 and β1-Integrin

Epithelial cell are attached to each other via cell-cell adhesion molecules while attached to substrate via other adhesions (Gumbiner, Cell 84:345-357 (1996); Le et al., Am J Physiol Cell Physiol 283:C489-499 (2002)). These cells could remodel their adhesion molecules and become migratory during pathological conditions including cancer metastasis and wound healing (Gumbiner, (1996) supra). In particular, integrin's are responsible for cell adhesion via their interaction with matrix proteins including fibronectin. Previously, it was shown that Arf6 regulate both endocytosis and recycling of β1-integrin, the major cell adhesion molecule (Powelka et al., 5:20-36 (2004); Li et al., Dev Cell 9:663-673 (2005)). Moreover, the crucial role of activated Arf6 in cell adhesion, migration and invasive phenotype of different breast cancer cells was also studies (Hashimoto et al., Proc Natl Acad Sci USA 101:6647-6652 (2004); Sabe, J Biochem 134:485-489 (2003)). In particular it was shown that activation of Arf6 by its GEF/BRAG2, which also directly interacts with epidermal growth factor receptor (EGFR), could induce breast cancer invasion and therefore contribute to its metastasis and malignancy (Morishige et al., Nat Cell Biol 10:85-92 (2008); Sabe et al., Cell Adh Migr 2:71-73 (2008)).

Cytohesin family proteins are also shown to be important in regulation actin rearrangement and cell motility (Frank et al., Mol Biol Cell 9:3133-3146 (1998); Klarlund et al., Science 275:1927-1930 (1997); Venkateswarlu et al., Biochem J 335 (Pt 1):139-146 (1998); Venkateswarlu et al., Curr Biol 8:463-466 (1998)). In particular, ARNO (cytohesin-2) was shown to modulate lamelliapodia formation and cell migration in MDCK kidney epithelial cells (Santy et al., J Cell Biol 154 (2001) 599-610 (2001); Geiger et al., EMBO J. 19:2525-2536 (2000)). Recently, the crucial and unique role of ARNO was also demonstrated for regulation of β1-integrin pathway. In particular, it was found that ARNO required for β1-integrin recycling and, therefore it is controlling cell adhesion. It was also shown that knockdown of ARNO and inhibition of its GEF-activity with Arf6 effectively inhibits cell spreading and migration (Oh et al., J Biol Chem 285:14610-14616 (2010)).

V-ATPase and β1-Integrin

Recently, the role of the V-ATPase in interaction with β1-integrin and in its processing have been also uncovered. It was demonstrated that c-subunit of V-ATPase directly interacts with β1-integrin and alter its glycosylation, which directly correlates with metastatic disease in a number of cancers (Skinner and Wildeman, J Biol Chem 274:23119-23127 (1999); Skinner and Wildeman, J Biol Chem 276:48451-4845 (2001); Li et al., J Immunol 180 (2008) 3158-3165 (2008)). It was also demonstrated that overexpression of c-subunit of V-ATPase in HEK293 cells inhibited cell migration and invasion (Skinner and Wildeman, J Biol Chem 276:48451-4845 (2001)). Similarly, overexpression of the c-subunit in HT1080 cells results in the inhibition of cell spreading in response to the fibronectin, the ligand of β1-integrin (Lee et al., J Biol Chem 279:53007-53014 (2004)). Importantly, the disruption of interaction between the c-subunit V-ATPase and β1-integrin prevent the inhibition of the invasiveness of these cells (Skinner and Wildeman, J Biol Chem 276:48451-4845 (2001); Li et al., J Immunol 180 (2008) 3158-3165 (2008)). Moreover, overexpression of c-subunit V-ATPase in HEK293, HT1080 and L6 also cells alters the surface expression of β1-integrin, indicating an important role of V-ATPase in its trafficking and recycling to the cell surface. It is noteworthy, that our studies also demonstrated that while a-subunit V-ATPase interact with ARNO, the c-subunit of V-ATPase directly interact with Arf6 small GTPase (Hurtado-Lorenzo et al., Nat Cell Biol 8:124-136 (2006)). Thus, the molecular mechanisms of modulation of the interactions within V-ATPase/ARNO/Arf6 complex, described herein, are also in position to regulate the function of β1-integrin on cell adhesion, motility and invasiveness.

Methods of Screening

Recently, a novel paradigm of “interfacial inhibition” for the discovery of drugs that interfere with macromolecular complexes has been proposed (Pommier and Cherfils, Trends in Pharmacol Sci 2005, 26: 138-145). The concept of “interfacial inhibition” is an alternative to “competitive inhibition” and takes advantage of the strategy for interfering with molecular interactions to trap macromolecular complexes (protein-protein, DNA-protein, etc) in transition states with their partners that are not able to complete their cell biological function. This concept is based on the mechanism of action of brefeldin A (BFA) a fungal macrolide that was originally isolated from Penicillum decumbens. This is a well known inhibitor of vesicular trafficking (Hurtado-Lorenzo et al., Nature Cell Biol 2006, 6:124-136), but the mechanism of its action was unknown. However, recent breakthroughs in structure-functional studies elucidated the mechanism action of BFA on the interface of the complex between an Arf small GTPase and the Sec7 domain of its Arf-GEF ARNO (Renauld et al., Nature 2003, 426; 525-530; Zeeh et al., J Biol Chem 2006, 281: 11805-11614). Use of this type of “interfacial inhibition” was proposed as a model for the therapeutic inhibition of GEFs in general (Zeghouf et al., Biochem Soc Trans 2005, 33: 1265-1268). Recently, the structure-based discovery of the novel inhibitor (called LM11) of Arf activation by Sec7 domains through targeting of protein-protein complexes was successfully performed using in silico screening of a flexible pocket near the Arf1/ARNO interface (Viaud et al., Proc Nat Acad Sci USA 2007, 104: 10370-10375). Also recently, another approach based on cytochesin-1 Sec7 domain fluorescence polarization assays gave rise to the discovery of a different small molecule (called SecinH3) which inhibits the Arf-GEF activity of cytochesin family members (Hainer et al., Nature Protocols, 2008, 3:579-587). Importantly, as discussed above inhibition of cytohesins by SecinH3 affected their association with the insulin-receptor, required for insulin signaling and results in hepatic insulin resistance (Hainer et al., Nature, 2006, 444:941-944; Fuss et al., Nature, 2006, 444:945-948; Jackson, Nature, 2006, 444:833-834).

Included herein are methods for screening test compounds, e.g., polypeptides, polynucleotides, inorganic or organic large or small molecule test compounds, to identify agents that interfere with the interaction between ARNO and the a2-subunit of V-ATPase. Such agents are useful in the treatment of disorders as described herein.

The methods described herein include assays for identifying compounds that interferes with the ARNO/V-ATPase interaction. The methods generally include providing a test sample, e.g., a cell-free test sample, that includes interacting domains from both ARNO (e.g., full-length ARNO or an isolated ARNO Sec7 domain) and a2-subunit V-ATPase (e.g., an isolated N-terminal cytosolic tail (a2N₁₋₄₀₂) or a2N-01 peptide). A test compound is introduced into the test sample, and binding between the interacting domains is evaluated. The level of binding between the interacting domains is compared to the level of binding in the absence of the test compound (e.g., in the same sample or in a control sample, or a reference level determined previously), and a decrease in binding in the presence of the test compound means that the compound is a candidate compound for inhibiting a2-subunit V-ATPase function and ARNO's GEF activity with Arf6.

In some embodiments, binding between the interacting domains is evaluated using one or both of fluorescence polarization (Hainer et al., Nature Protocols, 2008, 3:579-587, Chen et al., Biochem J., 2009, 420:283-294, Kawamoto et al., Biochemistry, 2009, 48:9534-9541) and plasmon resonance binding (e.g., BIAcore) assays, as described herein. Binding affinity determinations using surface plasmon resonance kinetic analysis between a2N-01 peptide and the Sec7-domain of ARNO gave a dissociation constant Kd=3.44×10⁻⁷ M; the binding affinity between wild-type a2N and the full length ARNO protein was Kd=3.13×10⁻⁷ M. These numbers can optionally be used as reference or control values in the binding assays described herein; a decrease in binding affinity in the presence of a test compound would indicate that the test compound inhibits the ARNO/V-ATPase interaction.

In some embodiments, to verify the ability of a compound that interferes with the ARNO/V-ATPase interaction to affect the activity of the complex, a candidate compound is applied to a test sample, e.g., a cell or living tissue or organ, e.g., and biochemical in vitro assays known in the art are used to test: i) enzymatic GEF-activity using purified proteins (Paris and Chabre, Biochemistry 2000, 39:5893-5901; Santy et al., Curr Biol 1999, 9:1173-1176), ii) enzymatic V-ATPase activity using purified proteins, endosomes and lysosomes (Marshansky et al., J Membr Biol 1996, 153:59-73; Marshansky and Vinay, Biochim Biophys Acta 1996, 1284:171-180; Kawashima et al., Kidney Int 1998, 54:275-278); iii) V-ATPase proton pumping/acidification using purified endosomes or lysosomes (Marshansky et al., J Membr Biol 1996, 153:59-73; Marshansky and Vinay, Biochim Biophys Acta 1996, 1284:171-180; Kawashima et al., Kidney Int 1998, 54:275-278); iv) cathepsins and legumain enzymatic activity using purified endosomes and lysosomes (Dando et al., Biochem J 1999, 339 (Pt 3):743-749; Alvarez-Fernandez et al., J Biol Chem 1999, 274:19195-19203; Trombetta et al., Science 2003, 299:1400-1403; Yamane et al., Biochim Biophys Acta 2002, 1596:108-120); and/or v) V-ATPase glucose-dependent assembly/disassembly using cell lysates (Trombetta et al., Science 2003, 299:1400-1403; Parra and Kane, Mol Cell Biol 1998, 18:7064-7074; Lu et al., J Biol Chem 2004, 279:8732-8739).

Cell biological assays known in the art can be used to test: i) uptake and trafficking of ligands (albumin and other proteins) via endosomal/lysosomal protein degradation pathway using fluorescent cell-population and single cell quantitative assay (Hurtado-Lorenzo et al., Nat Cell Biol 2006, 8:124-136; Slattery et al., Kidney Int 2008, 74:1480-1486); ii) protein degradation using fluorescent real-time-imaging and pulse-chase assays (Hurtado-Lorenzo et al., Nat Cell Biol 2006, 8:124-136); iii) megalin/cubilin-receptors trafficking and recycling assay (Hurtado-Lorenzo et al., Nat Cell Biol 2006, 8:124-136; Bose et al., J Physiol 2007, 581:457-466; Wolff et al., Toxicol Appl Pharmacol 2008, 230:78-85), iv) V-ATPase trafficking and recycling assay (Myers and Forgac, J Cell Physiol 1993, 156:35-42; Carraro-Lacroix et al., Pflugers Arch 2009, 458:969-979); v) autophagy detection and formation assay (Eng et al., Autophagy 2010, 6, In Press, PMID: 20458170; Proikas-Cezanne and Pfisterer, Methods Enzymol 2009, 452:247-260; Vazquez, Methods Enzymol 2009, 452:85-95; Seglen et al., Methods Enzymol 2009, 452:63-83); vi) cell adhesion assay (Kong et al., Proc Natl Acad Sci USA 2004, 101:10440-10445; Choesmel et al., Cancer 2004, 101:693-703; Liu et al., J Immunol Methods 2004, 291:39-49; Oh and Santy, J Biol Chem 2010, 285:14610-14616); vii) cell spreading assay (Oh and Santy, J Biol Chem 2010, 285:14610-14616; Porst et al., Kidney Int 2006, 69:450-456; Zeng et al., J Cell Biol 2003, 160:137-146); viii) cell migration assay (Oh and Santy, J Biol Chem 2010, 285:14610-14616; Porst et al., Kidney Int 2006, 69:450-456; Zeng et al., J Cell Biol 2003, 160:137-146); ix) scratch wound assay (Menon et al., Cell Motil Cytoskeleton 2009, 66:1041-1047; Walter et al., Exp Cell Res 2010, 316:1271-1281); x) cell invasiveness assay (Hinton et al., J Biol Chem 2009, 284:16400-16408; Albini and Benelli, Nat Protoc 2007, 2:504-511; Maliakal, Methods Enzymol 2002, 352:175-182; Hernandez et al., Methods Mol Biol 2009, 571:227-238); xi) beta1-integrin cell surface expression assay (Oh and Santy, J Biol Chem 2010, 285:14610-14616; Powelka et al., Traffic 2004, 5:20-36; Li et al., Dev Cell 2005, 9:663-673); xii) beta1-integrin trafficking and recycling assay (Oh and Santy, Biol Chem 2010, 285:14610-14616; Powelka et al., Traffic 2004, 5:20-36; Li et al., Dev Cell 2005, 9:663-673); and/or xiii) cytotoxicity assays (Guzman and McCrae, J Viol Methods 2005, 127:119-125; Zaritskaya et al., J Immunother 2009, 32:186-194).

Thus one or more effects of the test compound can be evaluated. In a living cell that expresses a functional V-ATPase/ARNO complex, for example, the ability of the test compound to inhibit a2-subunit V-ATPase activity and ARNO's GEF activity with Arf6 can be evaluated, e.g., by assaying using physiological assays to test: i) protein (e.g., albumin) uptake and degradation by kidney proximal tubules using two-photon confocal microscopy in live animals; ii) drug (e.g., gentamicin) uptake and lysosomal accumulation by kidney proximal tubules using two-photon confocal microscopy in live animals; iii) metastasis and formation of metastatic nodules using live animals; and/or iv) viral and bacterial cytotoxicity using live animals.

In some embodiments, the test sample is, or is derived from (e.g., a sample taken from) an in vivo model of a disorder as described herein. For example, an animal model, e.g., a rodent such as a mouse or rat, can be used.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

A test compound that has been screened by a method described herein and determined to inhibit binding between the ARNO and V-ATPase interacting domains, can be considered a candidate compound. A candidate compound that has been screened, e.g., in a living cell, and found capable of inhibiting of the enzymatic GEF-activity of ARNO and/or modulation of V-ATPase function within the V-ATPase/small GTPase complex, can be considered a candidate therapeutic compound for the treatment of a disease described herein. These candidate therapeutic compounds can then be evaluated using an in vivo model of a disorder, e.g., a cell or animal model of a disease described herein; candidate therapeutic compounds that are determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic compounds, candidate therapeutic agents, and therapeutic agents can be optionally optimized and/or derivatized, and formulated with physiologically acceptable excipients to form pharmaceutical compositions.

Thus, test compounds identified as “hits” (e.g., candidate compounds (that inhibit binding between the ARNO and V-ATPase interacting domains), and candidate therapeutic compounds (that is capable of inhibiting the enzymatic GEF-activity of ARNO and/or modulation of V-ATPase function within V-ATPase/small GTPase complex) can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of a disorder as described herein. The animal can be monitored for a change in the disorder, e.g., for an improvement in a parameter of the disorder, e.g., a parameter related to clinical outcome. In some embodiments, the subject is a mammal, e.g., a human.

Methods of Treatment

The methods described herein include methods for the treatment of disorders that would benefit from modulation of the function of V-ATPase or ARNO. Generally, the methods include administering a therapeutically effective amount of an a2N-01 peptide as described herein, to a subject who is in need of, or who has been determined to be in need of, such treatment.

V-ATPase as a Therapeutic Target

V-ATPase and its subunits have been suggested as an attractive targets for the treatment of variety of human genetic disorders, anti-cancer therapy as well as other human diseases (Bowman and Bowman, J Bioenerg Biomembr, 2005, 37: 431-435; Hikura Drug News Perspect., 2006, 19: 139-144).

a2-Subunit V-ATPase and Human Genetic Diseases

In the last few years, the V-ATPase was implicated in pathophysiology of variety of human diseases including some inherited genetic disorders (Marshansky and Futai, Curr Opin Cell Biol 2008, 20:415-426, Grüeber and Marshansky, BioEssays 2008, 30: 1096-1099, Forgac, Nat. Rev. Mol. Cell. Biol. 2007, 11: 917-29, Hinton et al., Pflugers Arch. 2009, 457: 589-598). Mutations in ATP6V0A2 gene encoding the human a2-subunit were implicated in Golgi function and development of congenital disorders of glycosylation in humans (Kornak et al., Nature Gen 2008, 40:32-34, Guillard et al., Biochim Biophys Acta, 2009, 1792: 903-914).

Cancer, Neurodegenerative, and Metabolic Diseases

Recently, small molecule V-ATPase inhibitors and siRNA were intensively studies with their potential application in cancer treatment (Lebreton et al., Bioorg Med Chem. Lett. 2008, 18: 5879-5883; Perez-Sayans et al., Cancer Treat Rev., 2009, 35: 707-713; You et al., Cancer Lett., 2009, 280: 110-119) and prevention of metastasis (Nikura et al., Cancer Chemother Pharmacol., 2007, 60: 555-562). The role of V-ATPase a-subunit isoforms (a1, a2, a3 and a4) dependent acidification has been studied in a variety of cancers and in invasive phenotype of metastatic tumors (Hinton et al., J. Biol. Chem. 2009, 284, 16400-16408, Hinton et al., Pflugers Arch. 2009, 457: 589-598). V-ATPase M8-9 subunit (ATP6AP2) and c-subunit (ATP6V0C) were implicated in regulation of Wnt signaling via LRP6 receptor in signalosomes (Cruciat et al., Science, 2010, 327: 459-463), regulation of caspase-independent apoptotic pathway (Sasazawa et al., Cancer Sci., 2009, 100: 1460-1467) and control of acidic tumor microenvironment (Hinton et al., J Biol. Chem., 2009, 284: 164000-16408). Thus, compounds identified by the methods described herein to inhibit the ARNO/V-ATPase interaction and inhibit V-ATPase activity can be used to reduce invasiveness (metastasis) of tumors. For example, the a2N-01 peptide (e.g., a2N-01-CPP fusion protein) can be administered to a subject who has metastatic cancer, or is at risk of developing metastatic cancer (e.g., has at least one tumor of a type considered likely to be invasive).

Bone Disease

Moreover, V-ATPase was proposed as a target in the treatment of lytic bone disorders including: i) osteoporosis, ii) Paget's disease, bone aseptic loosening and tumor-induced bone distruction (Xu et al., Histol. Histopathol., 2007, 22: 443-454). Moreover, the V-ATPase inhibitors were successfully used in prevention of experimental periodontitis (Nikura et al., J Toxic Sci., 2005, 30: 297-304; Nikura, J. Periodontol., 2006, 77: 1211-1216). Finally, the use of V-ATPase inhibitors war recently proposed as anti-influenza treatment (Gong et al., Curr Med. Chem., 2009, 16: 3716-3739).

Renal Disease

In kidney physiology, the receptor-mediated endocytosis by proximal tubule epithelial cells plays an important role in protein homeostasis via reabsorption of albumin, hormones, chemokines, vitamin-binding proteins and toxic drugs (Marshansky et al., Curr. Opin. in Nephrol. and Hyperten. 2002, 11:527-537; Brown and Marshansky, “The Renal V-ATPase: Physiology and Pathophysiology” in Handbook of ATPases: Biochemistry, Cell Biology, Pathophysiology (Futai et al., eds.) Wiley-VCH 2004:413-442; Wagner et al., Physiol. Rev. 2004, 84:1263-1314; Marshansky and Futai et al., Curr Opin Cell Biol 2008, 20:415-426; Grüeber and Marshansky et al., BioEssays 2008, 30: 1096-1099; Brown et al., J Exp Biol 2009, 212:1762-1772; Brown et al., Traffic 2009, 3:275-284; Hurtado-Lorenzo et al., Nature Cell Biol. 2006, 6:124-136). This endosomal/lysosomal protein degradation pathway also depends on V-ATPase dependent endosomal/lysosomal acidification, whose defects lead to proximal tubulopathies such as Dent disease, Fancony syndrome and drug toxicity in human and animal models (Id.).

Infection and Toxicology

The crucial role of V-ATPase and acidification in endocytosis of viruses, microorganisms and toxic molecules is generally accepted. The endocytotic pathway is used by viruses and bacteria to enter into the eukaryotic cells. They have developed the variety of strategies in order to reach their site of replication (cytosol or intracellular organelles) and/or to avoid their degradation in lysosomes (Gruenberg and Van der Goot, Nat Rev Mol Cell Biol 2006, 7:495-504; Huynh, Microbiol Molec Biol Rev 2007, 71: 452-462). One strategy of the infection process requires a V-ATPase-driven acidic environment in early or late endosomes and used by: i) stomatitis virus, ii) M2-protein from influenza A-virus and iii) SARS coronavirus. The translocation of various toxins from endosomes to cytosol also depends upon acidification and include among others: anthrax, diphtheria and clostridial toxins (Gruenberg and Van der Goot, Nat Rev Mol Cell Biol 2006, 7:495-504; Abrami et al., J Cell Biol 2004, 166: 645-651; Gilbert et al., FEBS Letters 2007, 581: 1287-1296).

An alternative strategy of survival is employed by Mycobacterium tuberculosis. These microorganisms diminish the acidity of phagosomes and impair their fusion with lysosomes (Gruenberg and Van der Goot, 2006, supra). It has been demonstrated that the human V-ATPase gene can protect or predispose the host to pulmonary tuberculosis (Capparelli et al., Genes Immun., 2009, 10: 641-646). In addition, V-ATPase was suggested as one of the novel vaccine candidates against Leishmania infection (Stober et al., Vaccine, 2006, 24: 2602-2616). Acidification-independent strategy of internalization is employed by HIV (human immunodeficiency virus) and requires the direct interaction with V-ATPase (Mandic et al., Mol Biol Cell 2000, 12: 463-473; Geyer et al., Mol Biol Cell 2002, 13: 2045-2056; Geyer et al., J Biol Chem 2002, 277: 28521-28529).

Arf Family Small GTPases and their Regulatory Proteins

The Ras-superfamily small GTPases function as “molecular switches” and regulate the extraordinary variety of cell functions (Bourne et al., Nature 1990, 348: 125-132). The transition between “on” and “off” states of this molecular device is mediated by GDP/GTP cycle. The ADP-ribosylation factor (Arf) family belongs to Ras-superfamily and also functions as molecular switch to regulate vesicular traffic and organelle structure (Donaldson and Klausner, Curr Opin Cell Biol 1994, 6: 527-532). Similar to other small GTPases, the activation of Arfs (GTP-bound conformation, “on”-state) is mediated by guanine-nucleotide exchange factors (GEFs), while desactivation (GDP-bound conformation, “off”-state) is catalyzed by GTPase-activating proteins (GAPs). All known Arf-GEFs contain a Sec7 conserved catalytic domain, which is responsible for GEF activity and is related to the prototype yeast protein Sec7p.

Arf-GEFs constitute a large and diverse family containing 49 proteins. ARF nucleotide-binding site opener (ARNO) also known as, cytohesin-2 (CYTH2) is a member of the cytohesin subfamily of Arf-GEFs, which includes another three members: cytohesin-1, cytohesin-3 (also called ARNO3 or GRP1) and cytohesin-4. Human cytohesins exhibit at least 80% similarity in a pairwise comparison of their amino acid sequences and share a common domain organization. Their structure is divided into the following four regions: i) an N-terminal coiled-coil (CC); ii) a central Sec7 domain; iii) a pleckstrin homology (PH) domain; and iv) a C-terminal polybasic (PB) motif region. The sequence of human ARNO is available in the GenBank database for cytohesin-2 isoform-1 under Accession Nos. NM_{—}017457.4 (nucleic acid) and NP_{—}059431.1 (amino acid) as well as under Accession Nos. NM_{—}004228.5 (nucleic acid) and NP_{—}004219.3 (amino acid), respectively, for cytohesin-2 iso form-2.

Both small GTPase Arf6 and its cognate GEFs have been implicated in regulation of endocytotic pathway and organelle biogenesis by: i) recruiting coat components; ii) modifying phospholipids; and iii) remodeling actin cytoskeleton near the vesicular membranes (Hurtado-Lorenzo et al., Nature Cell Biol 2006, 6:124-136; D'Souza-Schorey et al., Nat Rev Mol Cell Biol 2006, 7:347-358). Thus, the formation and regulation of V-ATPase/ARNO/Arf6 complex may regulate these downstream pathways. Moreover the interaction of V-ATPase/ARNO/Arf6 with aldolase (data not shown) strongly suggest that Arf small GTPases and ARNO can modulate the assembly/disassembly of the V-ATPase itself. The pH-sensing function of V-ATPase and acidification-dependent recruitment of small GTPases is an integral part of the glucose/aldolase-dependent regulation of V-ATPase itself. Thus, the generally accepted function of small GTPases as “molecular switches” may directly be applied to the assembly/disassembly of V-ATPase or turning “on/off” this nano-machine

Arf Family Small GTPase, their Regulatory GEF's Cytohesins and Diseases.

The transition of cells from attached to moving state is important in cell adhesion and cytokinesis and is significant for embryogenesis, wound repair, cancer pathogenesis and tumor cell invasion. Recent studies have demonstrated that Arf small GTPases and cytohesins play crucial roles in coordination of this transition (D'Souza-Schorey and Chavrier, Nat Rev Mol Cell Biol 2006, 7:347-358, Turner and Brown, Curr Biol., 2001, 11:R875-R877). In particular, Arf6 was shown to be involved in growth and invasiveness of various cancers including breast cancer (Hashimoto et al., Proc Natl Acad Sci USA, 2004, 101: 6647-6652), melanomas (Muralidharan-Chari et al., Cancer Res. 2009, 69, 2201-2209) and gliomas (Hu et al., Cancer Res., 2009, 69, 794-801). Tumor cells invasion and proliferation are regulated by Arf6 either via MEK/ERK (Tague et al., Proc Natl Acad Sci USA, 2004, 101:9671-9676) or epidermal growth factor (Morishige et al., Nat Cell Biol., 2008, 10:85-92; Valderrama and Ridley, Nat Cell Biol., 2008, 10: 16-18; Li et al., Cancer, 2009, 115: 4959-4972) signaling pathways. On the other hand, cytohesins and ARNO were recently identified as essential proteins in insulin signaling and development of diabetes (Hainer et al., Nature, 2006, 444:941-944; Fuss et al., Nature, 2006, 444:945-948; Jackson, Nature, 2006, 444:833-834).

These are some of the examples showing the crucial importance of V-ATPase/ARNO/Arf6 complex in cell biology as well as pathophysiology of human diseases.

### EXAMPLES

The invention is further described in the following examples, which do not limit the scope of the invention described in the claims.

### Example 1

### Mapping the Binding Sites on the N-Terminal Cytosolic Tail of the V-ATPase a2-Subunit (a2N) that are Involved in Interaction with ARNO

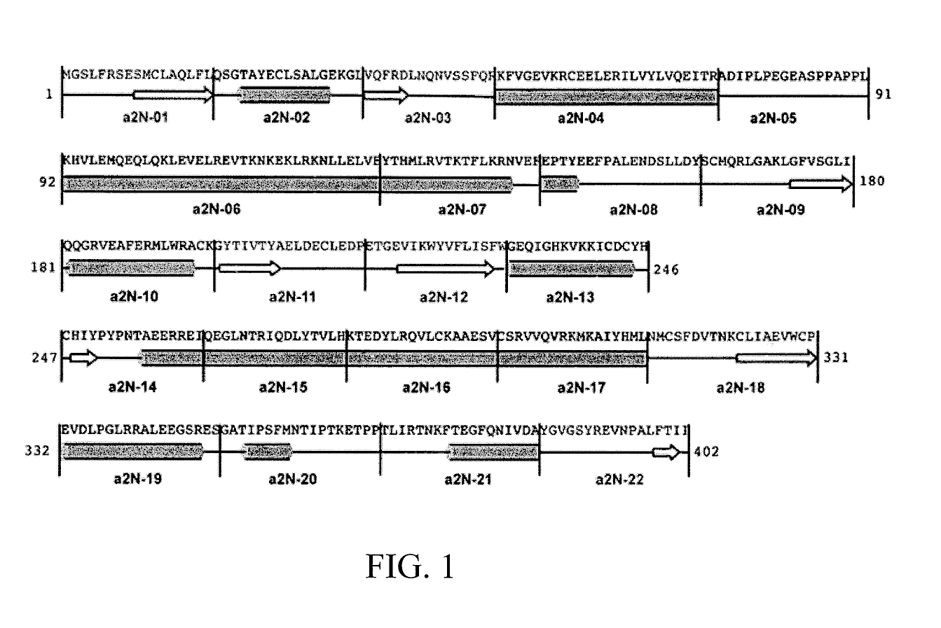

In order to map the a2N motifs that interact with ARNO, an a2N-derived peptide pull-down approach was applied. Since the three-dimensional structure of V-ATPase a-subunit isoforms is currently unknown, the rational design of a2N-derived peptides was performed using a prediction of the secondary structure for mouse a2N using the PSIPRED server (Jones, J Mol Biol 292 (1999) 195-202; McGuffin et al., Bioinformatics 16 (2000) 404-405). Moreover, the presence of coiled-coil domains was also predicted on a2N using COILS software (Lupas et al., Science 252 (1991) 1162-1164). According to this program, the mouse V-ATPase a2-subunit CC-domain spans from L92 to Y128. Using these two in silico prediction programs for guidance, 22 peptides covering the 1-402 amino acids of a2N were designed (FIG. 1A) and synthesized (Table 1). Recently, the involvement of CC-motifs in an interaction with syntaxin was demonstrated for the a1-subunit of V-ATPase from Drosophila melanogaster (Hiesinger et al., Cell 121 (2005) 607-620). Therefore, in order to test its possible involvement in interaction with ARNO the longest peptide a2N-06 (a2N₉₂₋₁₂₇) corresponding to the predicted CC-domain of a2N was also synthesized.

TABLE 1

| Peptide | AA of | # | AA Sequence of the synthesized | Seq id |
|---|---|---|---|---|
| Name | a2N | AA | peptides | no: |
| a2N-01 | 1-17 | 17 | Bio-MGSLFRSESMCLAQLFL-Cys * | 1 |
| a2N-02 | 18-34 | 17 | Bio-QSGTAYECLSALGEKGL-Cys | 2 |
| a2N-03 | 35-49 | 15 | Bio-VQFRDLNQNVSSFQR-Cys * | 3 |
| a2N-04 | 50-74 | 25 | Bio-KFVGEVKRCEELERILVYLVQEITR-Cys | 4 |
| a2N-05 | 75-91 | 17 | Bio-ADIPLPEGEASPPAPPL-Cys | 5 |
| a2N-06 | 92-127 | 36 | Bio-KHVLEMQEQLQKLEVELREVT | 6 |
| | | | KNKEKLRKNLLELVE-Cys | |
| a2N-07 | 128-145 | 18 | Bio-YTHMLRVTKTFLKRNVEF-Cys | 7 |
| a2N-08 | 146-163 | 18 | Bio-EPTYEEFPALENDSLLDY-Cys | 8 |
| a2N-09 | 164-180 | 17 | Bio-SCMQRLGAKLGFVSGLI-Cys | 9 |
| a2N-10 | 181-197 | 17 | Bio-QQGRVEAFERMLWRACK-Cys | 10 |
| a2N-11 | 198-214 | 17 | Bio-GYTIVTYAELDECLEDP-Cys * | 11 |
| a2N-12 | 215-230 | 16 | Bio-ETGEVIKWYVFLISFW-Cys * | 12 |
| a2N-13 | 231-246 | 16 | Bio-GEQIGHKVKKICDCYH-Cys | 13 |
| a2N-14 | 247-262 | 16 | Bio-CHIYPYPNTAEERREI-Cys | 14 |
| a2N-15 | 263-278 | 16 | Bio-QEGLNTRIQDLYTVLH-Cys | 15 |
| a2N-16 | 279-295 | 17 | Bio-KTEDYLRQVLCKAAESV-Cys | 16 |
| a2N-17 | 296-312 | 17 | Bio-CSRVVQVRKMKAIYHML-Cys | 17 |
| a2N-18 | 313-331 | 19 | Bio-NMCSFDVTNKCLIAEVWCP-Cys * | 18 |
| a2N-19 | 332-349 | 18 | Bio-EVDLPGLRRALEEGSRES-Cys | 19 |
| a2N-20 | 350-367 | 18 | Bio-GATIPSFMNTIPTKETPP-Cys | 20 |
| a2N-21 | 368-385 | 18 | Bio-TLIRTNKFTEGFQNIVDA-Cys | 21 |
| a2N-22 | 386-402 | 17 | Bio-YGVGSYREVNPALFTII-Cys * | 22 |

Bio - biotin tag. Cys - cysteine. The interacting peptides are indicated in bold with an
asterisk (*) and non-interacting peptides are in regular type face.

Peptides were synthesized, purified by HPLC and analyzed by mass spectrometry in the MGH Peptide/Protein Core Facility. All a2N-derived peptides were synthesized with a biotin-tag on the N-terminus for immobilization on streptavidin-agarose beads and with an additional C-terminal cysteine residue for immobilization on beads using sulfhydryl-specific crosslinkers. A peptide corresponding to the PB-domain of ARNO was synthesized containing either a non-phosphorylated or a phosphorylated Ser392 residue. Peptide stock solutions were prepared in DMSO at 5 mM concentration and stored at −20° C.

GST fusion protein pull-down assays were performed to study the direct interaction of recombinant proteins. The purified recombinant GST-ARNO(wt) and GST-tagged ARNO domain constructs (100 pmol) were incubated with 10 μl of in vitro translated 6His-a2N recombinant protein at 4° C. for 2 hours in 500 μl of binding buffer (10 mM HEPES, 1 mM EDTA, 1 mM DTT, 100 mM NaCl, 10% glycerol, 0.1% NP-40) with gentle rotation. Then the interacting recombinant proteins were immobilized on glutathione beads by incubation with 50 μl of 50% glutathione Sepharose 4B slurry for 2 hours at 4° C. The beads were collected by centrifugation and washed 5 times with 500 μl ice-cold binding buffer. Interacting proteins were eluted by boiling in SDS-PAGE sample buffer for 10 min at 95° C., resolved by SDS-PAGE and analyzed by Western blotting using polyclonal anti-a2N antibodies. Alternatively, pull-down experiments with GST-ARNO(wt) and GST-only as control were performed using in vitro translated and methionine-³⁵S metabolically labeled recombinant proteins a2N-N-[³⁵S] and a2N-C-[³⁵S], corresponding, respectively, to N-terminal (a2N₁₋₁₃₃) and C-terminal (a2N₁₃₄₋₃₉₃) parts of a2N. Pull-down experiments and analysis by autoradiography were performed as previously described (Hurtado-Lorenzo et al., Nat Cell Biol 8:124-136 (2006)). All experiments were repeated at least three times.

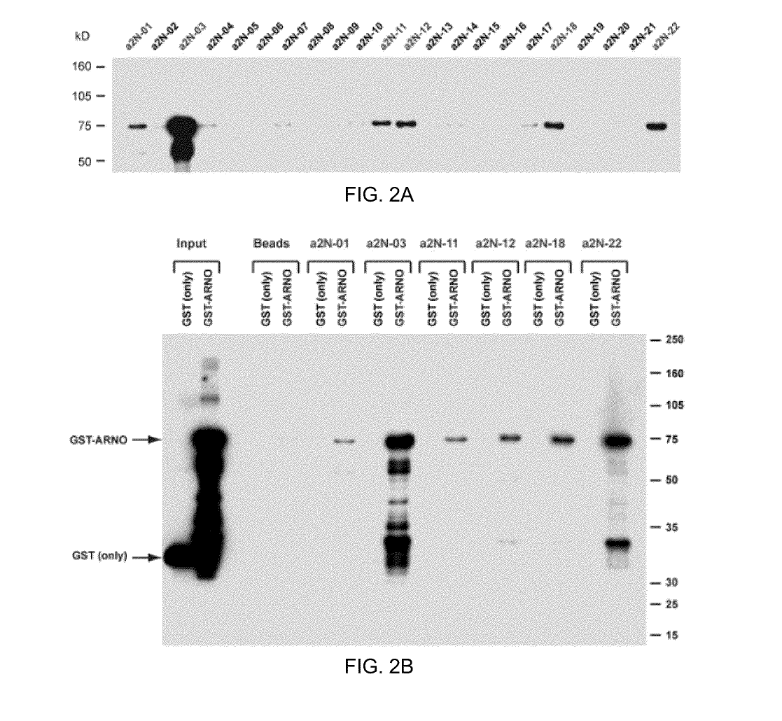

The peptide pull down experiments demonstrated a specific interaction of GST-ARNO(wt) with six of the a2N-derived peptides (a2N-01, a2N-03, a2N-11, a2N-12, a2N-18 and a2N-22) (FIG. 2A). The other 16 peptides, including a2N-06, which represents the putative CC-domain of a2N, did not bind to GST-ARNO(wt). The specificity of the GST-ARNO(wt) interaction was further confirmed using a GST-only construct as a control in the a2N-derived peptide pull-down assay (FIG. 2B).

In order to confirm the involvement of multiple sites on a2N in the interaction with ARNO, a recombinant protein pull-down assay was also used. For this purpose a2N was divided into two parts: i) a2N—N (or a2N₁₋₁₃₃), the N-terminal part which contains sequence covered by the peptides from a2N-01 to a2N-06; and ii) a2N-C (or a2N₁₃₄₋₃₉₃), the C-terminal part which contains sequence covered by the peptides from a2N-07 to a2N-22.

Constructs corresponding to amino acid residues 1-133 (a2N-N) and 134-393 (a2N-C) of the mouse V-ATPase a2-subunit were amplified using Expand High Fidelity PCR System and subcloned into a pIVEX2.4d vector (Roche, Indianapolis, Ind.) using NcoI/SmaI restriction sites. The resulting constructs contain a modified N-terminal 6×His-tag (MSGSHHHHHHSSGIEGRGRLIKMT (SEQ ID NO:23)). Both constructs were in vitro translated using the RTS 100 kit (Roche, Indianapolis, Ind.) and used in pull-down experiments. A construct corresponding to amino acid residues 1-402 of mouse V-ATPase a2-subunit (a2N₁₋₄₀₂) was amplified as above and subcloned into NdeI/NotI restriction sites of pET28b vector (Novagen, Gibbstown, N.J.) in frame with a thrombin cleavable N-terminal 6×His-tag. Recombinant a2N protein was expressed in E. coli BL21(DE3) cells and purified from inclusion bodies under denaturing conditions (in presence of 6 M Guanidine-HCL) on Talon beads (Clontech) and refolded into 1 M NDSB-256, 100 mM CHES-NaOH, pH 9.0, 1 mM DTT. Conditions for refolding were found by screening with iFOLD™ Protein Refolding System 2 (Novagen, Gibbstown, N.J.). After refolding, a2N protein was dialysed into 100 mM CHES-NaOH, pH 9.0, 1 mM DTT.

Cytohesin-2 or ARNO is a highly conserved protein with 99.25% identity between human and mouse species. There are only three conservative substitutions between human and mouse amino acids N86H, A124S and E229D in ARNO sequences. Thus, human ARNO was used in our experiments, since the crystal structure of its Sec7 domain in complex with Arf1 has been previously solved and was used in the modeling studies described herein. The following constructs corresponding to wild-type human ARNO (triglycine variant) and its domains were generated: i) 1-400aa, full length ARNO(wt); ii) 1-60aa, coiled-coil (CC) domain; iii) 61-252aa, Sec7 domain; iv) 253-378aa, plekstrin-homology (PH) domain; and v) 379-400aa, polybasic (PB) domain. All domains of ARNO were amplified with simultaneous incorporation of a 6×-His tag using Expand High Fidelity PCR System and subcloned into pGEX6P-1 vector (GE Healthcare, Piscataway, N.J.) using EcoRI/NotI restriction sites. The resulting constructs contain an N-terminal GST-tag and a C-terminal 6×His-tag. All five recombinant proteins were expressed in E. coli BL21(DE3) cells (Stratagene) and were purified by sequential chromatography on TALON beads (Clontech) and glutathione Sepharose 4B beads (GE Healthcare) according to the manufacturer's instructions. Alternatively, GST-tagged ARNO(wt) and its domains were purified by sequential chromatography on glutathione Sepharose 4B beads (GE Healthcare) and Superdex™ 200 HR 10/30 pre-packed column (GE Healthcare) using the “AKTA Purifier” system (GE Healthcare) according to the manufacturer's instructions. For BIAcore experiments (see Example 7, below), the GST-tag was cleaved from the Sec7 domain by PreScission Protease™ (GE Healthcare) according to the manufacturer's instructions.

These in vitro translated a2N—N and a2N—C mutants were used in pull-down experiments with purified GST-ARNO(wt) and the results, shown in FIG. 2C, demonstrated that both parts of a2N could interact with wild-type ARNO. Thus, the data indicate that while multiple interaction motifs of a2N are involved in the interaction with ARNO, its predicted CC-domain does not appear to be involved in this process.

### Example 2

### Homology Modeling of Human ARNO and Mapping the Binding Sites on ARNO that are Involved in Interaction with a2N

Cytohesin family members including ARNO contain the following distinct domains: i) CC-domain (aa 1-60); ii) Sec7-domain (aa 61-242); iii) PH-domain (aa 262-375) and iv) PB-domain (aa 380-400) as well as linkers: v) Sec7 and PH linker domains (aa 242-261) and vi) PH and PB linker domains (aa375-379) (Casanova, Traffic 8 (2007) 1476-1485; Chardin et al., Nature 384 (1996) 481-484). In order to properly define boundaries between these domains and regulatory elements, homology modeling of ARNO was performed. A spatial structure of human ARNO without the N-terminal CC region was made using the crystal structure of the autoinhibited form of Grp1 (also called ARNO3 or cytohesin-3)(PDB ID 2R09) as a template (DiNitto et al., Mol Cell 28 (2007) 569-583). In order to model full-length ARNO, the C-terminal domain of the human EB1 protein (PDB ID 2HKQ, chain A) was used as a template for the CC-domain of ARNO. Some dihedral angles in the chimeric template were manually edited in SWISS-PDB Viewer (Guex and Peitsch, Electrophoresis 18 (1997) 2714-2723) to avoid steric clashes. Energy minimization in Gromacs (Van Der Spoel et al., J Comput Chem 26 (2005) 1701-1718) was performed for equilibration of the template. The final models were built with Modeller9v2 (Marti-Renom et al., Annu Rev Biophys Biomol Struct 29 (2000) 291-325).

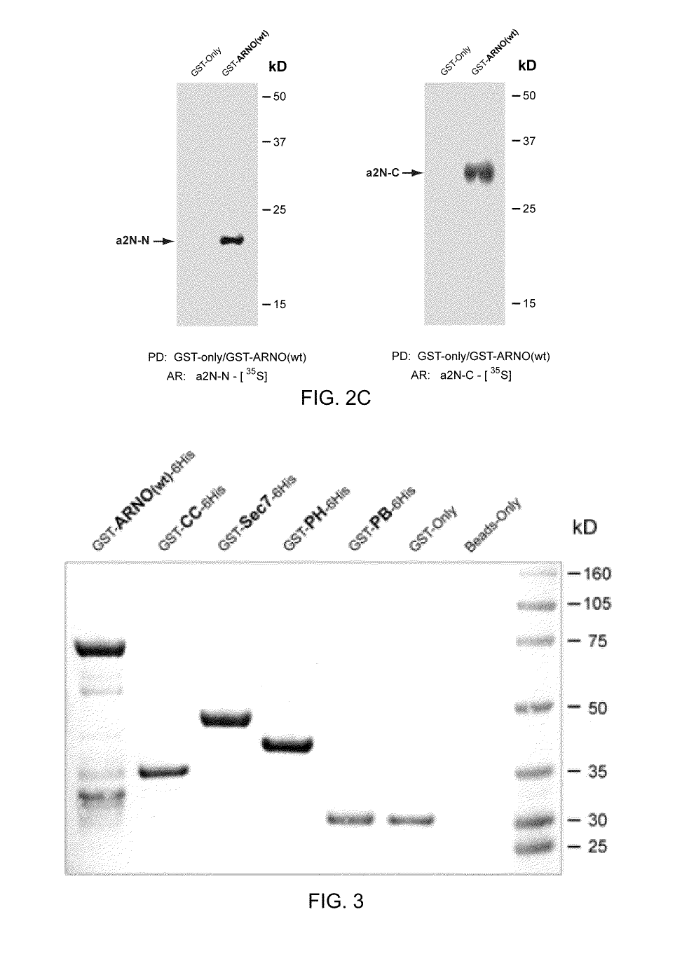

To examine the ability of these well-defined and functionally distinct domains of ARNO to interact with a2N₁₋₄₀₂, full-length ARNO(wt) and its four domains were cloned separately for bacterial expression of recombinant proteins (FIG. 3). The Sec7/PH-linker region (aa 242-261) was divided between the Sec7-domain and the PH-domain, while the PH/PB-linker region (aa 375-379) was cloned as a part of the PH-domain. Interaction of a2N with full-length ARNO(wt) was used as a control in these experiments. These purified recombinant proteins (FIG. 3) were used as baits in GST-pull-down experiments with in vitro translated recombinant a2N as a prey. Interacting proteins were detected by western blot analysis with a2-specific antibodies as described above.

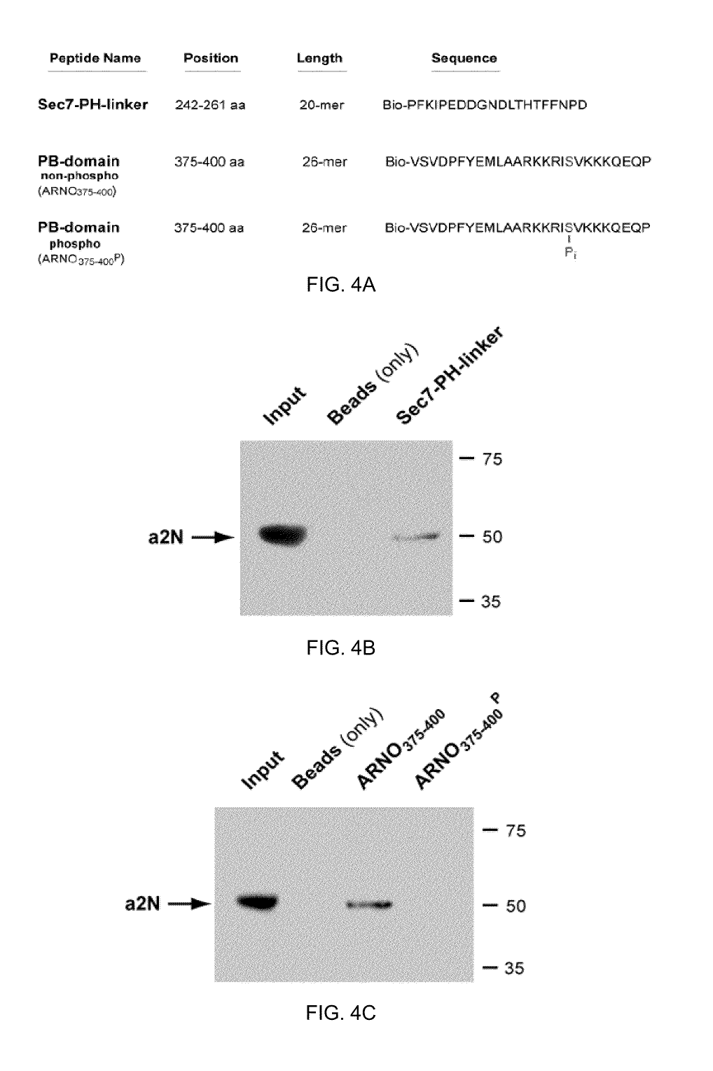

The data demonstrated that a2N interacts strongly with the ARNO Sec7-domain and weakly with its PH-domain, while no interaction with either the CC-domain or the PB-domain of ARNO could be detected. In order to determine whether the Sec7/PH-linker region itself could interact with a2N, a peptide corresponding to the Sec7/PH-linker region (242-261aa) of ARNO was synthesized (FIG. 4), and an interaction of a2N with this linker region was reproducibly observed. Thus, the protein GST pull-down and peptide pull-down assays demonstrated that a2N strongly interacts with ARNO's catalytic Sec7 domain, while weak binding to the PH-domain and the linker between these two domains was also detected.

### Example 3

### Phosphorylation-Dependent Interaction of a2N with the Regulatory PB-Domain of ARNO

The experiments described in this example tested whether: i) interaction of a2N with the PB-domain (379-400 aa, short 22 amino acids construct) was prevented by the addition of two tags (bulky 26 kDa GST-tag and His-tag) on the ARNO construct and ii) whether this interaction could be modified by Ser₃₉₂ phosphorylation of the PB-domain of ARNO. Thus, a biotinylated peptide called ARNO₃₇₅₋₄₄₀ which covers the PB-domain (379-440aa) and also includes four additional amino acids of a PH/PB-linker (375-378 aa) (FIG. 4A) was synthesized, along with a phosphorylated version of this peptide ARNO₃₇₅₋₄₄₀^{P} (the Ser₃₉₂-residue is known to be phosphorylated by PKC in vivo) was synthesized (FIG. 4A)(Santy, et al., Curr Biol 9 (1999) 1173-1176). These peptides were then used in pull-down experiments, as follows.

ARNO-derived peptides, equal amounts (7 nmol) of biotin-tagged synthetic peptides were also immobilized on streptavidin-agarose beads followed by incubation with 10 μl of in vitro translated 6His-a2N(wt) recombinant protein. Pull-down experiments and SDS-PAGE analysis were performed as described above and interacting 6His-a2N/ARNO-derived-peptide complexes were analyzed by Western blotting using polyclonal anti-a2N antibody. All experiments were repeated at least three times with similar results.

The results indicated that there was a direct interaction of a2N with non-phosphorylated ARNO₃₇₅₋₄₀₀ (FIG. 4C). Importantly, this interaction was specific since interaction between a2N and ARNO₃₇₅₋₄₄₀^{P} it was completely abolished by Ser₃₉₂-phosphorylation (FIG. 4C).

### Example 4

### Structural Changes of the ARNO₃₇₅₋₄₀₀ Peptide Caused by its Ser₃₉₂-Phosphorylation

In order to understand the phosphorylation-dependent mechanism of interaction between a2N and ARNO-derived peptides, the structural traits of the non-phosphorylated (ARNO₃₇₅₋₄₀₀) and phosphorylated (ARNO₃₇₅₋₄₀₀^{P}) peptides were determined by solution NMR spectroscopy. The non-phosphorylated (ARNO₃₇₅₋₄₀₀) and phosphorylated (ARNO₃₇₅₋₄₀₀^{P}) peptides (2 mM final concentration of each peptide) of ARNO were prepared by dissolving an appropriate amount in 50 mM phosphate buffer, pH 6.8. The one dimensional (1D) and two dimensional (2D) ¹H NMR spectra including total correlation spectroscopy (TOCSY) and nuclear Overhauser enhancement spectroscopy (NOESY) were obtained at a temperature of 288 K on an Avance Bruker NMR spectrometer at 700 MHz proton frequency. TOCSY and NOESY spectras of the peptide were recorded with mixing times of 80 and 300 ms, respectively. All the NMR data were processed using the Bruker Avance spectrometer built-in software Topspin. Peak-picking and data analysis of the Fourier-transformed spectra were performed with the SPARKY program (Kneller and Goddar, SPARKY 3.105., in: U.o.C.e. Edit (Ed.), San Francisco, Calif., 1997). Assignments were carried out according to classical procedures including spin-system identification and sequential assignment (Wüthrich, NMR of Proteins and Nucleic acids., Wiley, Interscience, New York, 1986). The three-dimensional structure of the peptides ARNO₃₇₅₋₄₀₀ and ARNO₃₇₅₋₄₀₀^{P} were calculated based on both distance and angle restraints by using the CYANA 2.1 program package (Herrmann et al., J Mol Biol 319 (2002) 209-227). Dihedral angle restraints were calculated from chemical shifts using torsion angle likelihood obtained from shift and sequence similarity (TALOS) (Wüthrich, NMR of Proteins and Nucleic acids., Wiley, Interscience, New York, 1986). In total twenty ARNO₃₇₅₋₄₀₀ and twenty ARNO₃₇₅₋₄₀₀^{P} structures were calculated.

All amino acids of the two peptides were sequentially assigned. The connectivity diagrams of ARNO₃₇₅₋₄₀₀ and ARNO₃₇₅₋₄₀₀^{p}, respectively, are indicative of a helical conformation with the sequential HN—HN, Ha—HN(i, i+3), Ha—HN(i, i+4), and Ha—Hb(i, i+3) connectivities. Data from assigned 2D NOESY spectra and primary amino acid sequence were used as input for the automated structure calculation using the Cyana 2.1 package (Herrmann et al., J Mol Biol 319 (2002) 209-227). In total, an ensemble of twenty calculated structures resulted in an overall mean root square deviation (RMSD) of 0.69 Å for non-phosphorylated ARNO₃₇₅₋₄₀₀ (PDB ID: 2 kpa and BMRB ID: 16550) and 0.378 Å for ARNO₃₇₅₋₄₀₀^{P} (PDB #: 2 kpb and BMRB #:16551). All these structures had energies lower than −100 kcal mol⁻¹, no NOE violations greater than 0.3 Å and no dihedral violations greater than 5°. Summary of the statistics for twenty structures are shown in the table. The calculated structure ARNO₃₇₅₋₄₀₀ forms a stable N-terminus region with a helix extending from 378-384, followed by a short loop from 385-387, a second helix between 388-391 and a third very unstable 3₁₀ helix from 394-397. The remaining C-terminal amino acids form a flexible structure. Structural regions of ARNO₃₇₄₋₃₉₉ are also reflected in the NOE plot. Electrostatic potential surface analysis showed that the peptide is strongly basic in nature, reflecting its name, the poly-basic (PB) domain of ARNO (Santy, et al., Curr Biol 9 (1999) 1173-1176). By comparison the structural assignment and calculation of ARNO₃₇₅₋₄₀₀^{P}, showed an N-terminus alpha-helix extending from residues 377-384 and a second alpha-helix at the C-terminus composed of residues 390-396, forming together a stable helix-loop-helix structure with a RMSD value of 0.378 Å. The loop between the two alpha-helices extends from residues 385-389. Superimposition of both structures, ARNO₃₇₅₋₄₀₀ ARNO₃₇₅₋₄₀₀^{P} via N-terminus 1-10 residues revealed a nice fitting in this region with a root mean square deviation of 0.68 Å but with difference in the C-terminus. An alignment of the structures at the N-termini resulted in a distance deviation of 105° between the two C-termini at the loop region of ARNO₃₇₅₋₄₀₀ ARNO₃₇₅₋₄₀₀^{P}, respectively. Thus, the Ser₃₉₂-phosphorylation of ARNO₃₇₅₋₄₀₀ peptide produced significant structural changes in the C-terminus, resulting in the disruption of the alpha-helix at the Ser₃₉₂ residue in the non-phosphorylated ARNO₃₇₅₋₄₀₀.

In contrast, ARNO₃₇₅₋₄₀₀^{P} displayed an extended helix through Ser₃₉₂ and attained a maximum stable structure. These structural changes could be responsible for the inhibitory effect of Ser₃₉₂-phosphorylation on ARNO₃₇₅₋₄₀₀ interaction with a2N observed in our peptide pull-down experiments as discussed below.

### Example 5

### Homology Modeling of ARNO Conformational Changes Caused by Ser₃₉₂-Phosphorylation of its PB-Domain

A spatial structure of human ARNO without the N-terminal CC region was derived using homology modeling as described above. The crystal structure of the autoinhibited form of Grp1 (PDB ID 2R09) was also used as a template and the model was built using Modeller9v2 software (Marti-Renom et al, Annu Rev Biophys Biomol Struct 29 (2000) 291-325). A spatial model of activated ARNO is shown in a complex with Arf6 and the structure was equilibrated in Gromacs (Van Der Spoel et al., J Comput Chem 26 (2005) 1701-1718). Nucleotide exchange on both Arf6 and Arf1 is activated by the Sec7 domain of ARNO. Superimposition of the known crystal structure of the human Sec7-Arf1 complex onto ARNO with Arf6 shows that the PH-domain and Arf6 occupy the same space. Thus, Arf6 binding may take place only concurrently with conformational changes in ARNO. First, since the exact relative positions between the Sec7 and PH-domains in activated cytohesins is currently unknown, during this step the PH-domain in the ARNO model was manually moved to a position that allows binding of Arf6. During the second step, the NMR structure of non-phosphorylated peptide ARNO₃₇₅₋₄₀₀ (PDB #: 2 kpa and BMRB #:16550) was used for modeling of the non-phosphorylated PB-domain in the ARNO model. On the other hand, the NMR structure of the phosphorylated peptide ARNO₃₇₅₋₄₀₀^{P} (PDB #: 2 kpb and BMRB #: 16551) was used for modeling of the phosphorylated PB-domain in the ARNO model. These modeling studies revealed the existence of a potential interaction pocket that is formed by the aC, aE and aG helices of the Sec7-domain and two semi-helices of the PB-domain of activated ARNO. This model also predicted that conformational changes caused by Ser₃₉₂-phosphorylation could result in the opening of this interaction pocket and, thus, modify its binding interfaces on both Sec7 and PB-domains of ARNO. Therefore, experiments were performed designed to identify the specific part of the a2N that may potentially be involved in the binding within this interaction pocket.

### Example 6

### Identification of a Motif on a2N that is Involved in Specific Binding with the Sec7 Domain of ARNO

In order to map the motif(s) on a2N that interact with the Sec7-domain of ARNO, the peptide pull-down approach was also applied using six of the a2N-derived ARNO-interacting peptides (a2N-01, a2N-03, a2N-11, a2N-12, a2N-18 and a2N-22).

These a2N-derived peptides were immobilized by an N-terminal biotin-tag, which ensured the same efficiency and equivalent binding to streptavidin-agarose beads. Simultaneous pull-down experiments with immobilized peptides were performed using highly purified recombinant GST-only or GST-Sec7-6His as described below. First, equal amounts (7 nmole) of the corresponding 6 synthetic peptides were immobilized on 25 μl of 50% streptavidin-agarose CL-4B (Fluka) slurry by mixing them overnight at 4° C. in 500 μl of binding buffer (10 mM HEPES pH 7.5, 1 mM EDTA, 1 mM DTT, 100 mM NaCl, 10% glycerol, 0.1% NP-40). To remove unbound peptides, beads were collected by centrifugation and washed in 500 μl ice-cold binding buffer. Next, equal amounts (20 pmoles) of highly purified recombinant GST-only or GST-Sec7-6His were added to the beads containing immobilized peptides and incubated for 2 hours at 4° C. in 500 μl of binding buffer. After washing beads four times in ice-cold binding buffer, the interacting complexes were eluted by boiling in SDS-PAGE sample buffer for 10 min at 95° C., resolved by SDS-PAGE and analyzed by Western blotting using monoclonal anti-GST antibody.

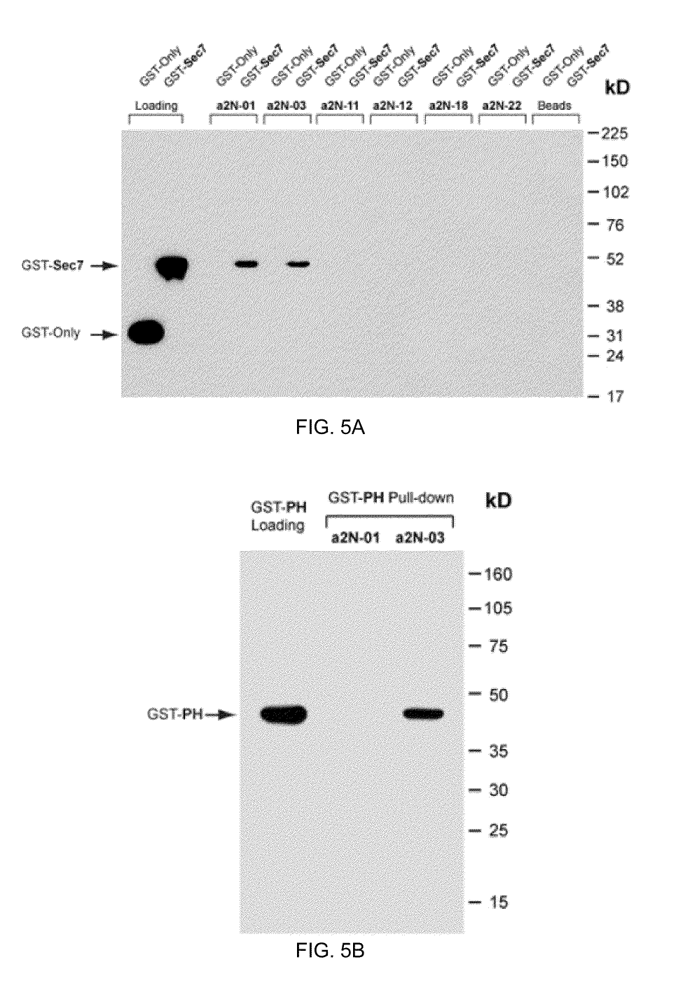

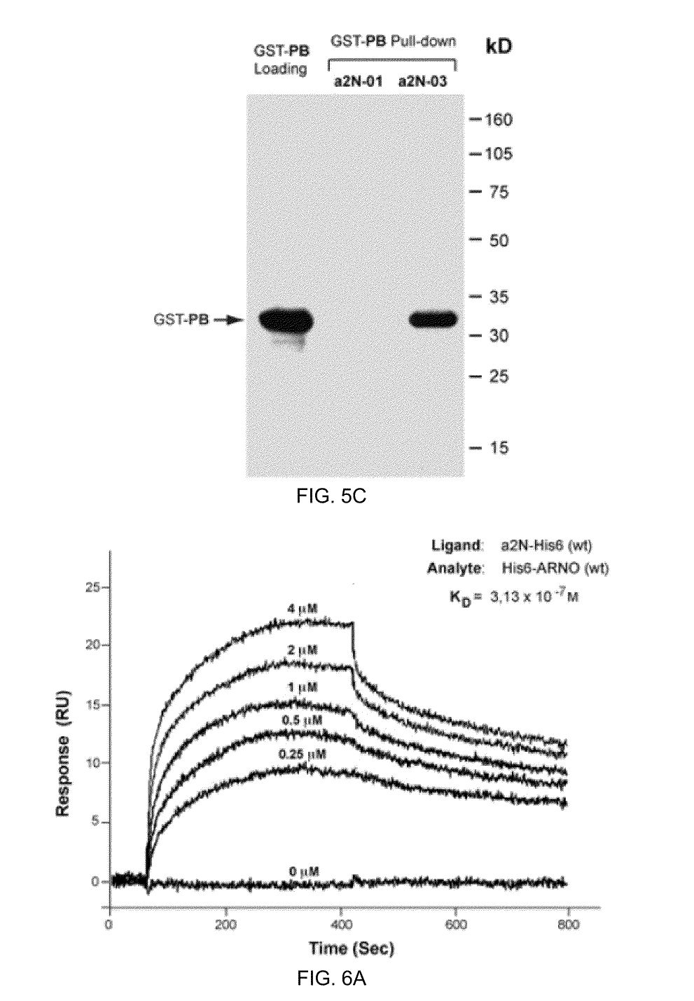

These experiments demonstrated that only two peptides a2N-01 and a2N-03 interact with the catalytic Sec7-domain of ARNO (FIG. 5A). Next, the specificity of binding of these two peptides to Sec7 was tested in comparison to other domains of ARNO. These pull-down experiments demonstrated that peptide a2N-01 (a2N₁₋₁₇, MGSLFRSESMCLAQLFL (SEQ ID NO:1)) showed specificity towards Sec7 domain and does not interact with either the PH-domain (FIG. 5B) or with the PB-domain (FIG. 5C) of ARNO. On the other hand, peptide a2N-03 lacks this specificity and interacts equally well with both the PH-domain (FIG. 5B) and PB-domain (FIG. 5C) of ARNO.

### Example 7

### Estimation of the Binding Affinities Using Surface Plasmon Resonance Analysis

To determine the dissociation constants (K_{D}) for binding of a2N(wt)/ARNO(wt) proteins and for the specific peptide a2N-01/Sec7-domain interaction, a kinetic analysis was performed using surface plasmon resonance (SPR) binding assays performed at 25° C. on a BIAcore™ T100 instrument (GE Healthcare). All reagents, including buffers, sensor chips and the amine coupling kit, were obtained from GE Healthcare. For kinetic analysis of the binding of ARNO(wt) with a2N(wt), purified 6His-a2N (5 μg/ml) in 10 mM sodium acetate (pH 4.0) was immobilized at 80 response units (RU) on a CM4 sensor chip using an amine coupling kit according to the manufacturer's instructions. The same kit was used to perform blank immobilization to create a reference surface on the same chip. Samples of purified GST-ARNO(wt) at concentrations ranging from 0.25-4 μM were injected for 6 min over active and reference surfaces at a flow rate of 30 μl/min in NBS-EP, 1 mM DTT running buffer (10 mM Hepes, pH 7.4, 150 mM NaCl, 3 mM EDTA, 0.05% Surfactant P20, 1 mM DTT). For kinetic analysis of the binding of the ARNO Sec7-domain with a2N-01 peptide, neutravidin (10 μg/ml) in 10 mM sodium acetate (pH 5.0) was immobilized at about 1200 RU on two surfaces of a CM4 sensor chip using an amine coupling kit. Following that, biotinylated a2N-01 peptide (50 RU) and amine-PEG₃-biotin (25 RU) were irreversibly captured by neutravidin to create active and a reference surfaces, respectively. Purified Sec7-6His recombinant protein was injected for 3.5 min over active and reference surfaces at concentrations ranging from 0.05-1 μM and a flow rate of 100 μl/min in NBS-EP, 1 mM DTT running buffer. Dissociation of the complexes was monitored for 10 min, and then the regeneration of the sensor surfaces was performed using 0.05% SDS with 1-min injection in the running buffer at a 20 μl/min flow rate. BIAcore™ T100 Evaluation software was used to calculate the association and dissociation rate constants (kₒₙ and k_{off}) with a 1:1 fitting model. The dissociation constant (K_{D}) was determined from the kₒₙ/k_{off} values. To estimate affinity of interaction the corresponding free energy of binding ΔG_{bind}=RT1nK_{D} was calculated as previously described (Marles et al., Mol Cell 14 (2004) 813-823).

Specific concentration-dependent responses were obtained for both interactions. When various concentrations of His6-ARNO(wt) were applied to a2N-His6(wt) immobilized on a sensor chip, a specific real-time binding between the two proteins was observed. The best fitted curves with bivalent analyte interaction isotherms were obtained and calculation of K_{D} was performed using the BIAcore™ T100 Evaluation software. An association rate constant (kₒₙ) of 4.77×10³ M⁻¹S⁻¹ was obtained and a dissociation rate constant (k_{off}) of 14.95×10⁻⁴ S⁻¹ as determined, giving a dissociation constant of K_{D} (k_{off}/kₒₙ) for this interaction of K_{D}=3.13×10⁻⁷ M. The kinetic analysis and estimation of K_{D} for the a2N-01/Sec-7 interaction was also calculated using a2N-01 peptide immobilized as ligand on the sensor chip, with various concentrations of Sec7-domain as analytes. The best fitted curve was a single 1:1 interaction isotherm and kₒₙ was determined as 2.5×10³M⁻¹S⁻¹ and k_{off} was determined as 8.6×10⁻⁴ S⁻¹ with a calculated K_{D} for this interaction of 3.44×10⁻⁷M. For both interactions, association rates were fast and dissociation rates were slow, which resulted in the formation of a relatively stable complex that was readily detectable in our pull-down (FIGS. 2A-2C and FIG. 5A) and previous immuno-precipitation experiments (Hurtado-Lorenzo et al., Nat Cell Biol 8:124-136 (2006)). Calculated free energies for a2N(wt)/ARNO(wt) and a2N-01/Sec7 binding are −8.86 kcal mol⁻¹ and −8.81 kcal mol⁻¹, respectively (Marles et al., Mol Cell 14 (2004) 813-823). These equivalent free energies strongly indicate that specific binding between peptide a2N-01 in the a2N and the Sec7 domain of ARNO is most likely one of the major contributors to the high affinity interaction between a2N and ARNO.

### Example 7

### Specific Intracellular Delivery of Tagged a2N-01 Peptide: Targeting to and Modulation of Endosomal/Lysosomal Protein Degradation Pathway

V-ATPase may not simply recruit and scaffold small GTPases to the target membrane during its function as a pH-sensing receptor. Rather, via interaction with the catalytic and regulatory elements of ARNO, V-ATPase could have a role as a modulator of the GEF activity of ARNO, ultimately regulating the activity of the Arf family of small GTPases in a broader context.

To test this hypothesis, the following water-soluble peptides of a2N-01 were synthesized that were tagged either with PEG or TAT peptide sequences and labeled with either Biotin or FITC.

| | | | SEQ |
|---|---|---|---|
| | | | ID |
| Experiment | Title | Sequence | NO: |
| Peptide | PEG-a2N- | PEG-MGSLFRSESMCLAQLFL | 1 |
| Synthesis #1: | 01 | | |
| Peptides | Biotin- | Biotin-PEG-MGSLFRSESMCLAQLFL | 1 |
| labeling #1: | PEG-a2N- | | |
| | 01 | | |
| | FITC- | FITC-βA-PEG-MGSLFRSESMCLAQLFL | 1 |
| | PEG-a2N- | | |
| | 01 | | |
| Peptide | a2N-01- | MGSLFRSESMCLAQLFL-PEG | 1 |
| Synthesis #2: | PEG | | |
| Peptides | Biotin- | Biotin-MGSLFRSESMCLAQLFL-PEG | 1 |
| labeling #2: | a2N-01- | | |
| | PEG | | |
| | FITC- | FITC-βA-MGSLFRSESMCLAQLFL-PEG | 1 |
| | a2N-01- | | |
| | PEG | | |
| Peptide | TAT-a2N- | YGRKKRRQRRRMGSLFRSESMCLAQLFL | 24 |
| Synthesis #3: | 01 | | |
| Peptides | Biotin- | Biotin- | 24 |
| labeling #3: | TAT-a2N- | YGRKKRRQRRRMGSLFRSESMCLAQLFL | |
| | 01 | | |
| | FITC- | FITC-βA- | 24 |
| | TAT-a2N- | YGRKKRRQRRRMGSLFRSESMCLAQLFL | |
| | 01 | | |
| Peptide | a2N-01- | MGSLFRSESMCLAQLFLYGRKKRRQRRR | 25 |
| Synthesis #4: | TAT | | |
| Peptides | Biotin- | Biotin- | 25 |
| labeling #4: | a2N-01- | MGSLFRSESMCLAQLFLYGRKKRRQRRR | |
| | TAT | | |
| | FITC- | FITC-βA- | 25 |
| | a2N-01- | MGSLFRSESMCLAQLFLYGRKKRRQRRR | |
| | TAT | | |

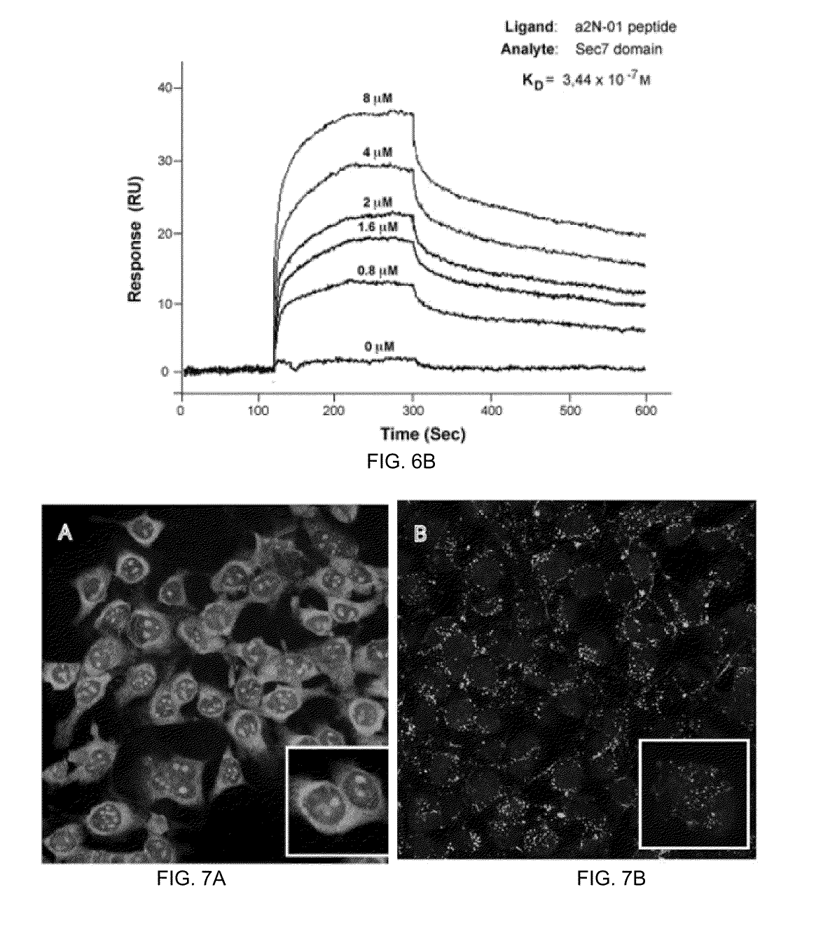

Intracellular delivery of the CPP-peptide fusions was assayed as follows. MTC cells were grown to 50% confluency in DMEM/FBS on 4 chamber slides. Cell penetrating peptides either 5 μM FITC-TAT or 5 μM FITC-a2N-01-TAT were dissolved in Ringer buffer (122.5 mM NaCl, 5.4 mM KCl, 1.2 mM CaCl₂, 0.8 mM MgCl₂, 0.8 mM Na₂HPO₄, 0.8 mM NaH₂PO₄, 5 mM Glucose, 10 mM Hepes, pH=7.4) and incubated with MTC cells for 10 minutes at 37° C. Cells were then washed three times with Ringer buffer followed by 20 minutes fixation in 4% PFA. Slides were mounted with in 2:1 mixture of Vectashield mounting medium in 1.5 M Tris solution (pH 8.9). Confocal microscopy analysis was performed on a Bio-Rad Radiance 2000 confocal microscope using LaserSharp 2000 software and images were reconstructed using Volocity v.5 software. FIGS. 7A-B show the specific and efficient delivery of cell penetrating fluorescent peptide FITC-a2N-01-TAT to intracellular vesicular compartments of MTC cells.

Live-real-time confocal microscopy of trafficking of FITC-a2N-01-TAT peptide and albumin-Alexa594 in MTC cells was also performed using a spinning disk UltraVIEW RS confocal imaging system and analyzed using Volosity v.5 software. These experiments demonstrated the targeting of both FITC-a2N-01-TAT and albumin-Alexa555 to the endosomal/lysosomal protein degradative pathway and their significant colocalization with overlap coefficient R=0.994.

### Example 8

### Specific and Efficient Inhibition of Enzymatic GEE-Activity of ARNO with Arf6 Small GTPase

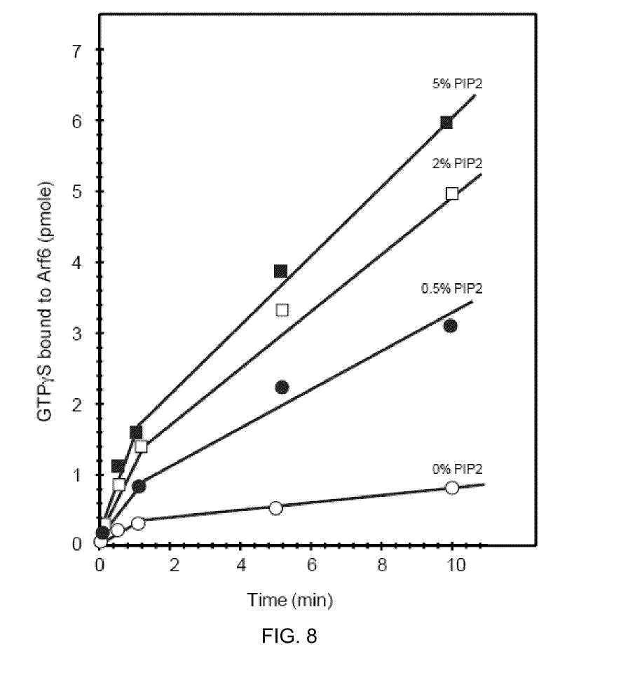

To test the effect of V-ATPase-derived peptides on enzymatic activity of small GTPases, the GDP/GTP exchange activity assay of ARNO with Arf6 was established in the presence of GTPγS and phospholipids vesicles containing various amounts of phosphatidylinositol PIP2 (FIG. 8).

Phospholipid vesicles were prepared by the extrusion method (Macia and Paris Biochemistry, 39: 5893-5901 (2000)). The lipids were dissolved in chloroform. A film of phospholipids was formed by evaporating chloroform and was resuspended in 50 mM Hepes, pH 7.5, and 1 mM dithiothreitol. The suspension was freeze-thawed 3 times and was passed through a 0.1 μm pore size polycarbonate filter.

GDP/GTP-exchange activity of ARNO was measured using time-course radiolabel based assay as previously described (Santy et al., Curr Biol. 9:1173-1176 (1999)). Briefly, recombinant myristoylated Arf6 was diluted to 1 μM in a buffer containing 50 mM Hepes, pH 7.5, 1 mM MgCl₂, 100 mM KCl, 1 mM dithiothreitol and 1 mg/ml liposomes containing: phosphatidylcholine (PC) 65% (w/w), phosphatidylserine (PS 30% (w/w) and indicating amounts (w/w) of phosphatidylinositol-4,5-bisphosphate (PIP2). Reactions were initiated by addition of 50 nM ARNO and 4 μM [³⁵S]GTPγS and incubated at 37° C. At indicated time aliquots (20 μl) were diluted into 2 ml of stop buffer (20 mM Hepes, pH 7.5, 10 mM MgCl₂, 100 mM KCl). Proteins were immobilized on nitrocellulose filters by filtration. After washing filters 3 times with 2 ml of stop buffer, radioactivity was quantified After washing filters 3 times with 2 ml of stop buffer, radioactivity was quantified using Perkin-Elmer “Tri-Carb 2900TR” liquid-scintillation counter.

In peptide-inhibition experiments, 500 nM of recombinant ARNO were incubated in the presence of different amounts (0-2000 nM) of FITC-a2N-01-TAT peptide or FITC-TAT peptide as a control. Incubation of ARNO with peptides took place at room temperature for 10 minutes and GDP/GTP-exchange reaction was initiated by mixing with 1 μM myristoylated Arf6 also pre-incubated in the presence of 1 mg/ml liposome containing 65% (w/w) PC, 30% (w/w) PS, 5% (w/w) PIP2 and 4 μM [³⁵S]GTPγS. The enzymatic activities were followed for 30 min at 37° C. and radioactivity of aliquots were analyzed as described above.

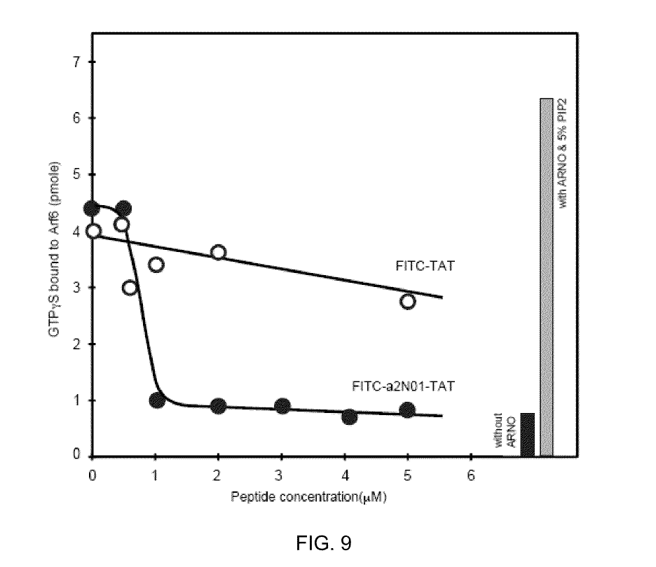

Confirming the hypothesis, specific inhibition of ARNO's GEF activity was seen with Arf6 by synthetic peptide FITC-a2N-01-TAT but not by control peptide FITC-TAT (FIG. 9).

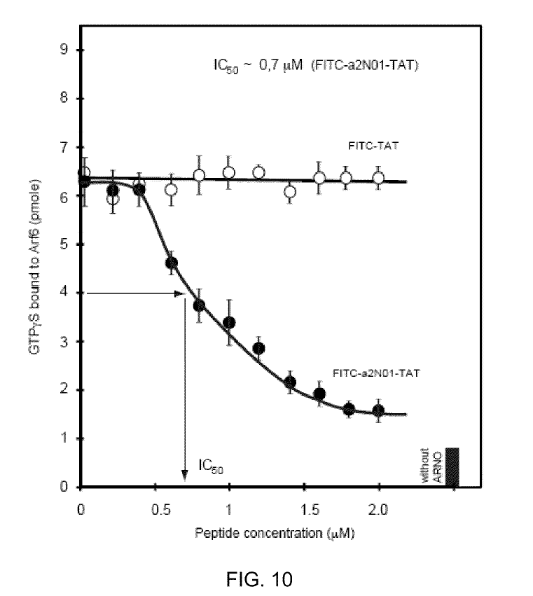

An IC₅₀ of about 0.7 μM was determined for this peptide, showing the potent and specific inhibition of ARNO's GEF activity by V-ATPase-derived peptide FITC-a2N-01-TAT (FIG. 10), confirming its high affinity and specific interaction with ARNO's Sec7 domain as well as full-length ARNO and a2N₁₋₄₀₂ seen in the BIAcore experiments (see Example 7, above). Importantly, in the above-described experiments on ARNO's GEF-activity the entire a2N₁₋₄₀₂ recombinant protein was also successfully used to modulate GDP/GTP-exchange activity of ARNO.

### Example 9

### Modulation of the Function of Endosomal/Lysosomal Pathway by FITC-a2N-01-TAT Cell Penetrating Peptide

To study the effect of cell penetrating FITC-a2N-01-TAT peptide on the megalin/cubilin-dependent endocytosis of albumin-Alexa 555 in MTC cells the fluorimetric cell-population assay was applied as previously described (Hurtado-Lorenzo et al., Nat Cell Biol 8:124-136 (2006)).

Briefly, MTC cells were plated at 5% confluency, grown for 7 days for 100% confluency and polarization and were starved for 2 days in FBS-free DMEM medium. At the day of experiment cells were washed in Ringer buffer and pre-incubated with either 5 μM of FITC-a2N-01-TAT peptide or 5 μM of FITC-TAT peptide as a control for 10 min. The albumin uptake by monolayer of MTC cells as initiated by adding of albumin-Alexa555 at 100 μg/well corresponding to the final concentration 200 μg/ml. At indicated times the uptake was stopped by adding of 500 μl of ice-cold Ringer buffer and washed three times on ice with the same buffer. After washing cells were permeabilized with 500 μl of MOP's buffer containing 0.1% Triton X100 and uptake of albumin-Alexa 555 was quantified using Beckman Coulter “DTX 880” multimode detector.

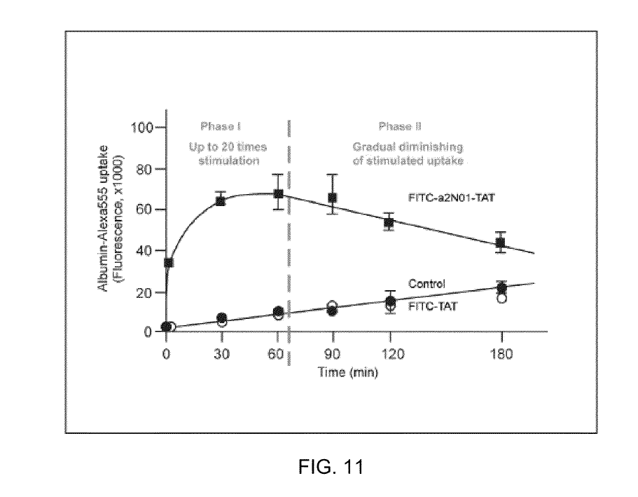

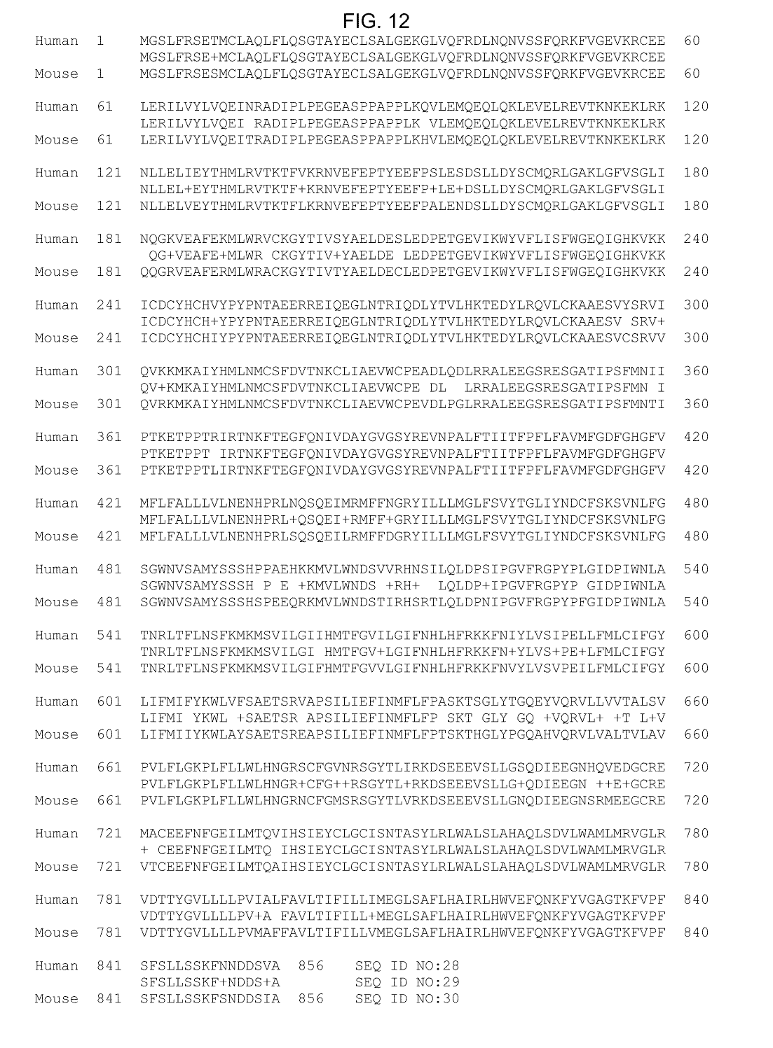

These uptake experiments with Alexa-555-labeled albumin in living mouse proximal tubules cells showed that vesicular trafficking within the endosomal/lysosomal protein degradation pathway is greatly modified by the FITC-a2N-01-TAT peptide, but not by a control FITC-TAT peptide lacking the a2N-01 sequence. See FIG. 11.

Based on this experimental data, the a2N-01 peptide is useful as an agent for the inhibition of ARNO GEF-activity in the V-ATPase/ARNO/Arf6 complex and modulation of endosomal/lysosomal protein degradation pathway in living cells. Other compounds that disrupt the ARNO Sec7/a2-subunit of V-ATPase interaction, and that are capable of inhibiting of the enzymatic GEF-activity of ARNO and modulation of V-ATPase function within V-ATPase/small GTPase complex and thus would also be expected to modulate vesicular trafficking can be identified using the methods described herein.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated peptide of SEQ ID NO:27.

2. A fusion peptide comprising (i) a vacuolar-type H+-ATPase (V ATPase)-derived sequence consisting essentially of SEQ ID NO:1 and (ii) a non-V-ATPase sequence.

3. The fusion peptide of claim 2, wherein the non-V-ATPase sequence is a cell-penetrating peptide.

4. A method of identifying candidate compounds that inhibit binding of a2-subunit V-ATPase to ARF nucleotide-binding site opener (ARNO) the method comprising:
providing a test sample comprising
(i) an ARNO interacting domain comprising Sec7 region of ARNO; and
(ii) an a2-subunit V-ATPase interacting domain comprising an isolated a2N peptide or ARNO-binding fragment thereof;
contacting the test sample with a test compound;
determining a level of binding between the interacting domains in the presence of the test compound; and
comparing the level of binding between the interacting domains to a level of binding in the absence of the test compound, wherein a decrease in binding in the presence of the test compound means that the test compound is a candidate compound that inhibits binding of a2-subunit V ATPase to ARNO.

5. The method of claim 4, wherein the a2-subunit V ATPase interacting domain comprises a2N-01 (or a2N₁₋₁₇).

6. The method of claim 4, wherein the level of binding is determined using plasmon resonance binding.

7. The method of claim 4, wherein the interacting domains each comprise a fluorescent moiety, and the level of binding is determined using fluorescence polarization.

8. The method of claim 4, further comprising:
selecting a candidate compound that inhibits binding of a2-subunit V-ATPase to ARNO;
providing a cell that expresses a functional a2-subunit V-ATPase/ARNO complex;
contacting the cell with the selected candidate compound;
evaluating activity of one or both of the a2-subunit V-ATPase and ARNO in the cell, and
selecting as a candidate therapeutic compound a candidate compound that inhibits activity of one or both of the ARNO and a2-subunit V-ATPase in the cell.

9. The method of claim 8, wherein ARNO's GEF activity with Arf6 is assayed using a radiolabel-based GDP/GTP-exchange assay.

10. The method of claim 8, wherein vesicular trafficking is assayed using megalin/cubilin-mediated uptake of labeled albumin

11. The method of claim 10, wherein uptake is assayed using a fluorimetric cell-population assay.


SEQUENCE LISTING (30 sequences)

SEQ ID NO: 1 - protein, 17 aa, Artificial Sequence
  Synthetic Peptide
MGSLFRSESMCLAQLFL

SEQ ID NO: 2 - protein, 17 aa, Artificial Sequence
  Synthetic Peptide
QSGTAYECLSALGEKGL

SEQ ID NO: 3 - protein, 15 aa, Artificial Sequence
  Synthetic Peptide
VQFRDLNQNVSSFQR

SEQ ID NO: 4 - protein, 25 aa, Artificial Sequence
  Synthetic Peptide
KFVGEVKRCEELERILVYLVQEITR

SEQ ID NO: 5 - protein, 17 aa, Artificial Sequence
  Synthetic Peptide
ADIPLPEGEASPPAPPL

SEQ ID NO: 6 - protein, 36 aa, Artificial Sequence
  Synthetic Peptide
KHVLEMQEQLQKLEVELREVTKNKEKLRKNLLELVE

SEQ ID NO: 7 - protein, 18 aa, Artificial Sequence
  Synthetic Peptide
YTHMLRVTKTFLKRNVEF

SEQ ID NO: 8 - protein, 18 aa, Artificial Sequence
  Synthetic Peptide
EPTYEEFPALENDSLLDY

SEQ ID NO: 9 - protein, 17 aa, Artificial Sequence
  Synthetic Peptide
SCMQRLGAKLGFVSGLI

SEQ ID NO: 10 - protein, 17 aa, Artificial Sequence
  Synthetic Peptide
QQGRVEAFERMLWRACK

SEQ ID NO: 11 - protein, 17 aa, Artificial Sequence
  Synthetic Peptide
GYTIVTYAELDECLEDP

SEQ ID NO: 12 - protein, 16 aa, Artificial Sequence
  Synthetic Peptide
ETGEVIKWYVFLISFW

SEQ ID NO: 13 - protein, 16 aa, Artificial Sequence
  Synthetic Peptide
GEQIGHKVKKICDCYH

SEQ ID NO: 14 - protein, 16 aa, Artificial Sequence
  Synthetic Peptide
CHIYPYPNTAEERREI

SEQ ID NO: 15 - protein, 16 aa, Artificial Sequence
  Synthetic Peptide
QEGLNTRIQDLYTVLH

SEQ ID NO: 16 - protein, 17 aa, Artificial Sequence
  Synthetic Peptide
KTEDYLRQVLCKAAESV

SEQ ID NO: 17 - protein, 17 aa, Artificial Sequence
  Synthetic Peptide
CSRVVQVRKMKAIYHML

SEQ ID NO: 18 - protein, 19 aa, Artificial Sequence
  Synthetic Peptide
NMCSFDVTNKCLIAEVWCP

SEQ ID NO: 19 - protein, 18 aa, Artificial Sequence
  Synthetic Peptide
EVDLPGLRRALEEGSRES

SEQ ID NO: 20 - protein, 18 aa, Artificial Sequence
  Synthetic Peptide
GATIPSFMNTIPTKETPP

SEQ ID NO: 21 - protein, 18 aa, Artificial Sequence
  Synthetic Peptide
TLIRTNKFTEGFQNIVDA

SEQ ID NO: 22 - protein, 17 aa, Artificial Sequence
  Synthetic Peptide
YGVGSYREVNPALFTII

SEQ ID NO: 23 - protein, 24 aa, Artificial Sequence
  Synthetic Peptide
MSGSHHHHHHSSGIEGRGRLIKMT

SEQ ID NO: 24 - protein, 28 aa, Artificial Sequence
  Synthetic Peptide
YGRKKRRQRRRMGSLFRSESMCLAQLFL

SEQ ID NO: 25 - protein, 28 aa, Artificial Sequence
  Synthetic Peptide
MGSLFRSESMCLAQLFLYGRKKRRQRRR

SEQ ID NO: 26 - protein, 17 aa, Artificial Sequence
  Synthetic Peptide
MGSLFRSETMCLAQLFL

SEQ ID NO: 27 - protein, 17 aa, Artificial Sequence
  Synthetic Peptide
MGSLFRSEXMCLAQLFL

SEQ ID NO: 28 - protein, 16 aa, Homo sapiens
  Synthetic Peptide
SFSLLSSKFNNDDSVA

SEQ ID NO: 29 - protein, 14 aa, Artificial Sequence
  Synthetic Peptide
SFSLLSSKFNDDSA

SEQ ID NO: 30 - protein, 16 aa, Mus musculus.
SFSLLSSKFSNDDSIA